(19) United States
(12) Patent Application Publication
(10) Pub. No.: US 2024/0268720 A1
(43) Pub. Date: Aug. 15, 2024

(54) WEARABLE DEVICE WITH FUNCTION OF DETERMINING HEMOGLOBIN CONCENTRATION, METHOD AND SYSTEM FOR DETERMINING HEMOGLOBIN CONCENTRATION

(71) Applicant: Samsung Electronics Co., Ltd., Suwon-si (KR)

(72) Inventors: Vladislav Valerievich LYCHAGOV, Saratov (RU); Vladimir Mikhailovich SEMENOV, Malakhovka (RU); Elena Konstantinovna VOLKOVA, Saratov (RU); Dmitrii Igorevich CHERNAKOV, Cherepovets (RU)

(21) Appl. No.: 18/434,212

(22) Filed: Feb. 6, 2024

(30) Foreign Application Priority Data
Feb. 9, 2023 (RU) 2023102891

(51) Int. Cl. A61B 5/1455; A61B 5/00; G16H 40/67; G16H 50/20

(52) U.S. Cl. CPC A61B 5/14551; A61B 5/681; A61B 5/7253; G16H 40/67; G16H 50/20

(57) ABSTRACT

A wearable device and a method for determining a hemoglobin concentration in a user's blood are provided. The method includes irradiating a tissue of a user with radiation of at least two different wavelengths, detecting photoplethysmographic (PPG) signals from the user's tissue in a time domain at the at least two different wavelengths in a reflection mode, transforming the detected PPG signals from the time domain to a frequency domain or a time-frequency domain, describing the transformed PPG signals with respect to harmonics to obtain a set of characteristics, and determining a hemoglobin concentration in the user's blood based on the obtained set of characteristics and an information from a database.

### CROSS-REFERENCE TO RELATED APPLICATION(S)

This application is based on and claims priority under 35 U.S.C. § 119(a) of a Russian patent application number 2023102891, filed on Feb. 9, 2023, in the Russian Intellectual Property Office, the disclosure of which is incorporated by reference herein in its entirety.

### BACKGROUND

### 1. Field

The disclosure relates to devices and systems for non-invasive, personal and/or on-demand monitoring of human health, in particular determining a total concentration of hemoglobin (Hb) as well as its various forms, such as oxyhemoglobin, deoxyhemoglobin, methemoglobin, carboxyhemoglobin, and other properties of blood, for example, oxygenation. More particularly, the disclosure relates to a method for determining a hemoglobin concentration, implemented in wearable devices, in particular in modern smartwatches and fitness bracelets.

### 2. Description of Related Art

Hemoglobin is a main respiratory pigment and a main component of red blood cells, performing important functions in the human and animal body, transfer of inhaled oxygen from lungs to biological tissues and organs, and transfer of carbon dioxide from the tissues and organs to the lungs, where it is exhaled. This occurs due to that oxygen is able to be reversibly bound by iron, the atoms of which are “built-into” hemoglobin. When interacting with biological tissues and organs, red blood cells are “released” from oxygen and “take away” carbon dioxide. Hemoglobin also plays a significant role in maintaining the acid-base balance of blood. The buffer system created by hemoglobin facilitates pH of blood to be maintained within certain limits.

One red blood cell (erythrocyte) contains about 3.4×10⁸ hemoglobin molecules, each of which consists of about 103 atoms. Human blood in average contains about 14.5% of hemoglobin, its total amount is about 750 g. Hemoglobin is a complex protein belonging to the group of hemoproteins, the protein component of which is represented by globin, the non-protein component—by prosthetic group. The prosthetic group in the hemoglobin molecule is represented by 4 identical ferroporphyrin compounds, which are referred to as hemes. The heme molecule consists of porphyrin IX bound to iron by two nitrogen atoms with covalent bonds and by two other nitrogen atoms by coordination bonds. The iron (II) atom is located in the heme center and imparts distinctive red color to the blood, its oxidation state does not change regardless of the addition or release of oxygen.

As discussed above, the most characteristic property of hemoglobin is the reversible addition of gases, such as oxygen (O₂), carbon dioxide (CO₂), or the like. The resulting compounds are referred to as oxyhemoglobin and carboxyhemoglobin respectively. The reaction of addition of molecular oxygen is not true oxidation of hemoglobin, since the valence of iron in heme does not change, and this reaction is more correctly referred to as oxygenation. True oxidation of hemoglobin occurs only when iron passes into a trivalent state.

In blood, hemoglobin exists in at least four forms, oxyhemoglobin, deoxyhemoglobin, carboxyhemoglobin, methemoglobin. In erythrocytes (red blood cells), molecular forms of hemoglobin are capable of interconversion, their ratio is determined by individual particulars of body.

Normal hemoglobin concentration values are, 130-165 g/l in men, 120-150 g/l in woman, 110-155 g/l in children, 110-120 g/l in pregnant women. Diagnostically significant decrease in the lower threshold of normal hemoglobin levels occurs in men in the age group of 65-74 years.

Hemoglobin concentration (level) increases when a number of red blood cells increases, and decreases if they are becoming fewer. A decrease in a number of red blood cells, and therefore, a reduced hemoglobin concentration in blood (low hemoglobin) may be caused by reducing the formation of red blood cells in the bone marrow, their loss as a result of bleeding or destruction inside the body, unbalanced nutrition, bad habits, impaired hemoglobin synthesis, disorders of gastrointestinal tract, chronic disorders of kidneys, hepatic cirrhosis, myxedema, hemolysis, or the like. The consequences of low hemoglobin are failure of the immune system, worsen skin condition, negative affect on the ability to motherhood, tachycardia, shortness of breath, provoking a stroke and heart attack. Meanwhile, a human is often in a state of anemia, when the body does not receive enough oxygen, which causes weakness, drowsiness and rapid fatigability.

An increased hemoglobin concentration is indicative of polycythemia, hemoconcentration during anhydration, burns, intestinal obstruction, persistent vomiting, stay at high altitudes, excessive physical exertion, cardiovascular pathology usually being congenital and leading to significant venous discharge, lung diseases leading to a decrease in pulmonary perfusion, poor aeration of the lungs, pulmonary arterial fistula, chronic chemical exposure to nitrites, sulfonamides, which cause the formation of meth- and sulfohemoglobin.

Saturation of blood with oxygen (oxygenation or SpO₂) is related to hemoglobin concentration, which is percentage of oxyhemoglobin in blood, i.e., the amount of oxyhemoglobin to total amount of hemoglobin ratio. For example, oxygenation of 95-100% is normal, but may vary if a human has lung disorder. Oxygenation <90% is considered to be low and this is referred to as hypoxemia, wherein the body requires oxygen supplementation. The reasons for low blood oxygen saturation are anemia, sleep apnea, smoking, lung disorders (asthma, pulmonary emphysema, or the like), viral infections (COVID-19, or the like), air quality, decreased capacity of heart, strong painkillers. The consequences of low oxygenation are headaches, tachycardia, shortness of breath, heart and brain damage. It should be noted that the oxygenation level reflects the degree of saturation of blood with oxyhemoglobin, but does not characterize the total hemoglobin content in the blood.

Thus, the hemoglobin concentration and the saturation of human blood with oxygen are among the most important and significant indicators of human health and are required to be constantly monitored.

Invasive and non-invasive methods are used to determine hemoglobin. Invasive methods are laboratory methods, which include chemical, spectrophotometric, colorimetric methods, such as the saponin method, the Sahli method, or the like, when hemoglobin derivatives formed during its oxidation and addition of various chemical groups to the heme are most often analyzed, which result to a change in an iron valence and a solution color. Chemical and spectrophotometric methods are highly accurate and are recommended as reference ones, but due to the labor intensity and significant cost of analysis, they are not used for routine determinations. For routine laboratory tests, the colorimetric methods are most preferred, as the cheapest, simplest and fastest to perform. The arterial blood gas test (ABG test) is used to determine blood oxygen saturation.

At the same time, methods for monitoring hemoglobin and blood oxygen saturation associated with blood sampling and testing are not suitable for continuous monitoring and are inconvenient. The advantages of measuring a hemoglobin concentration using wearable devices, in particular, smartwatches or fitness bracelets, are a non-invasive measurement method, quick measurement, ease of use, the possibility of continuous and long-term hemoglobin monitoring. In addition, a rapid oxygen saturation test can be useful for continuous monitoring and making a decision on the patient's hospitalization.

Pulse oximetry, for example, which provides control of the percentage of oxygen (O₂) saturated hemoglobin by assessing tissues for transmission of optical radiation (by pulse amplitude) and heart rate relates to non-invasive methods. For non-invasive determination of blood oxygenation, a tissue portion containing arterial vessels is placed in a working area of a photoplethysmographic (PPG) sensor. Traditional oximeters are usually in the shape of “clip” (clothespin) for placement onto a finger, earlobe of a patient, i.e., where radiation can be transmitted through a tissue sample, and analyze the radiation passing through the finger, earlobe, i.e., measure the tissue sample for transmission. In human tissues, bones, venous blood and arterial blood, radiation is absorbed, reflected and scattered, which, in the test of blood flow, is determined by the size of blood vessels or the volume of blood passing through the tested tissue portion. Vessel narrowing and expanding under the action of arterial pulsation of blood flow, which is mainly caused by changes in blood flow in the arteries and arterioles, cause a corresponding change in an amplitude of a signal received from the output of the photodetector. The pulse oximetry technique is based on the use of the principles of photoplethysmography (PPG). Hemoglobin serves as a kind of a filter for a light flux, wherein its “color” and “thickness” can vary. The “color” of the filter depends on the percentage of oxyhemoglobin. The capability of a pulse oximeter to derive a blood oxygenation degree is based on this. The change in the “thickness” of the filter is affected by the increase in blood volume in the arteries and arterioles with each pulse wave. After the pulse rate and pulse wave amplitude are measured, the ratio of the absorption degree of radiation of different wavelengths, for example, IR and red waves is analyzed by using a microprocessor, then a saturation of the pulsating arterial blood flow with oxygen is calculated.

Meanwhile, a signal from the sensor output, proportional to the absorption of light passing through the tissues, includes two components, a variable (alternating) component (AC) caused by change in arterial blood volume with each heartbeat, and a constant (direct) component (DC) determined by optical properties of skin, bones, venous blood and other tissues of the tested portion, the total absorption of which does not change during the propagation of the pulse wave.

This is the traditional approach for analyzing a signal in the pulse oximetry, which is based on separation of the constant and variable components of the time series (DC and AC components in a time domain or the components of direct and alternating currents) of the PPG signal (pulse signal). Meanwhile, the integral area of a curve of the signal is traditionally measured, and the waveform is usually not taken into account. At the same time, the DC component is used to normalize (condition) the signal, since the transmission of the signal depends not only on the absorption/reflection/scattering coefficients of skin, bones, blood and other tissues that make up the “filter” for the signal, but also on the thickness of the “filter” itself.

Most of the known methods describe the theory and operation of pulse oximeters in a transmission mode, therefore, in prior art, such approaches are described based on the relevant laws and equations, in particular, the Bouguer-Lambert-Behr law, which determines the attenuation of a parallel monochromatic beam of light when it propagates in an absorbing medium. However, there is insufficient information in prior art regarding the application of the above method with AC/DC components in a pulse oximeter in a reflection mode. Meanwhile, it is obviously that the operation of a device in the reflection mode requires other approaches, including it further requires an analysis of the pulse wave shape.

However, there are certain difficulties in analyzing AC components of a PPG signal, consisting in that the AC component has a complex shape that depends on a wavelength and carries an information about spectral properties of a tissue.

Devices for determining hemoglobin have low accuracy and are sensitive to motion artifacts, and therefore, they are not suitable for using as consumer gadgets. In addition, pulse oximeters, for example, have an additional device, i.e., a clip on a finger and are not suitable for continuous monitoring of blood oxygen saturation.

A patent U.S. Pat. No. 8,255,028 B2 (Masimo Corporation) discloses a patient monitor that has multiple sensors being capable of attaching to tissue sites of a living subject. The sensors generate sensor signals that are responsive to at least two wavelengths of optical radiation after being attenuated by pulsatile blood within the tissue sites. A patient monitor uses a plurality of signals to reduce the effect of noise.

The drawbacks of this solution are the non-use of information about a pulse wave shape, the lack of correction for scattering/absorption properties of the tissue.

A patent U.S. Pat. No. 9,341,565 B2 (Masimo Corporation) discloses a physiological monitor for determining blood oxygen saturation of a medical patient, including a sensor, a signal processor and a display. The sensor includes at least three light-emitting diodes. Each light-emitting diode is capable of emitting a light of a different wavelength. The sensor also includes a detector capable of receiving the light from the at least three light-emitting diodes after being attenuated by a tissue. The detector generates an output signal based at least in part on the received light. The signal processor determines blood oxygen saturation based on at least the output signal, and the display provides an indication of the blood oxygen saturation.

The drawbacks of this solution are also the non-use of information about a pulse wave shape, the lack of correction for the scattering/absorption properties of the tissue.

Table 1 showing the comparative particulars of the proposed disclosure and other solutions known in the prior art is shown below.

TABLE 1

| | | | mHematology | HemaApp | Fingernale |
|---|---|---|---|---|---|
| | | | https:// | http:// | https://doi.org/ |
| | | Cercacor | doi.org/ | dx.doi.org/ | 10.1038/ |
| | Proposed | https:// | 10.1364/ | 10.1145/ | s41467-018- |
| Characteristic | device | www.cercacor.com/ | OPTICA.390409 | 2971648.2971653 | 07262-2 |
| Presence of | No | Yes, finger | No | Yes, light | No |
| auxiliary | | clip | | source | |
| device | | | | | |
| Performance | High | Medium | Medium | Low | Low |
| Power | Low | High | Low | High | Low |
| consumption | | | | | |
| Continuous | Possible | Not | Not | Not | Not |
| monitoring | | possible | possible | possible | possible |
| Motion | High | Medium | Extremely | Very | Very |
| resistance | | | low | low | low |

The prior art solutions generally provides for the operation of sensors in a transmission mode and do not comprehensively disclose the principles of operation of wearable devices with function of determining blood properties, such as hemoglobin concentration, in a reflection mode, which is used, for example, in the form factor of smartwatches.

For example, recent publications, such as WO 2023/003980 A1 and WO 2023/287789 A1 (Masimo Corporation) disclose wearable devices in the form of smartwatches for monitoring physiological parameters of a user, that can be placed on a wrist, with groups of radiation sources and detectors located on the same side of the devices. At the same time, when describing the physical principles of operation of a PPG sensor, the authors of these publications refer to the Bouguer-Lambert-Behr law, as noted above with respect to the other prior art solutions. However, when operating in a reflection mode, this approach does not work and provides an incorrect result. In addition, signal processing is performed by traditional pulse oximetry signal processing methods, i.e., the focus of these publications is on an arrangement of the sensor, without describing specific signal processing methods, especially with regard to measuring specific blood components, such as hemoglobin. In a standard mode, when the sensor is within a watch on a hand, as a rule, it is possible to measure the pulse rate, its variability and oxygenation. In order to measure specific parameters, an external sensor connected to the watch port and placed, for example, on a user's hand finger can be used. Thus, the above publications do not disclose the particulars of processing signals received in a reflection mode, which allow for measuring specific blood parameters, such as a hemoglobin concentration. For example, in these publications there is no information about shapes of the PPG signal at different wavelengths, the difference in optical paths of radiation at these wavelengths, the difference in types of vessels and tissues with which radiation of different wavelengths predominantly interacts, and accordingly about a way to take into account the differences in optical paths and types of vessels and tissues via the shape of the PPG signal, and about the method for describing the shape of this signal by means of amplitude-phase characteristics of harmonics.

Thus, the main problem of this technical field consists in the lack of a method and an easy-to-use wearable device for continuously monitoring blood properties, for example, a hemoglobin concentration, especially when sensors are operated in a reflection mode, and also in the instability of existing solutions to motion artifacts.

Hence, there is a need for a wearable device and a method for determining blood properties, in particular, a hemoglobin concentration, which provide increased spectral resolution and being suitable for non-professional use, i.e., a reliable and motion resistant solution is required. In other words, a device for non-invasive, unobtrusive, continuous monitoring of a hemoglobin concentration and blood oxygen saturation is required.

Possible products in which the method according to the disclosure is used are wearable devices, such as smartwatches or smart bracelets, stationary diagnostic tools, household appliances and gadgets for personal medical monitoring.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide wearable devices with function of determining a hemoglobin concentration in a user's blood based on data collected from sensors of the wearable devices.

Another aspect of the disclosure is to provide a wearable device, method and system providing the possibility of personal, non-professional use, motion resistance and the capability to determine blood properties, in particular a hemoglobin concentration with high accuracy.

Additional aspects will be set forth in part in the description which follows and, in part, will be apparent from the description, or may be learned by practice of the presented embodiments.

In accordance with an aspect of the disclosure, a wearable device with a function of determining a hemoglobin concentration in a user's blood is provided. The wearable device includes at least one photoplethysmographic (PPG) sensor configured to irradiate a tissue of the user with radiation of at least two different wavelengths and to detect at least two PPG signals at the at least two different wavelengths in a reflection mode, wherein the wearable device is configured to transform the at least two PPG signals from a time domain to a frequency domain or time-frequency domain, describe the transformed PPG signals with respect to harmonics to obtain a set of characteristics, and determine a hemoglobin concentration in the user's blood based on the obtained set of characteristics and an information from a database.

In accordance with another aspect of the disclosure, a method for determining a hemoglobin concentration in a user's blood is provided. The method includes irradiating a tissue of a user with radiation of at least two different wavelengths, detecting photoplethysmographic (PPG or pulse) signals from the user's tissue in a time domain at the at least two different wavelengths in a reflection mode, transforming the detected PPG signals from the time domain to a frequency domain or a time-frequency domain, describing the transformed PPG signals with respect to harmonics to obtain a set of characteristics, and determining a hemoglobin concentration in the user's blood based on the obtained set of characteristics and an information from a database.

In accordance with another aspect of the disclosure, a system for determining a hemoglobin concentration in a user's blood is provided. The system includes a wearable device, and a remote server and/or a cloud storage, wherein the system comprises a communication interface between the wearable device and the remote server and/or the cloud storage.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of the certain embodiments of the disclosure will be more apparent from the following description taken in conjunction with the accompanying drawings, in which:

FIG. 1 schematically illustrates a dependence of a photoplethysmographic (PPG) waveform (a shape of a PPG signal) on a wavelength and a user according to an embodiment of the disclosure;

FIG. 2A schematically illustrates a difference in mechanisms of scattering and absorption of radiation on a probe volume in a transmission mode and a reflection mode according to an embodiment of the disclosure;

FIG. 2B schematically illustrates PPG signal shapes in a transmission mode and a reflection mode according to an embodiment of the disclosure;

FIG. 3A schematically illustrates absorption spectra of whole blood at different concentrations of hemoglobin according to an embodiment of the disclosure;

FIG. 3B schematically illustrates absorption spectra of a tissue (blood+other components) at a same concentration of hemoglobin according to an embodiment of the disclosure;

FIG. 4A schematically illustrates a first traditional approach to PPG signal processing according to an embodiment of the disclosure;

FIG. 4B schematically illustrates a second traditional approach to PPG signal processing according to an embodiment of the disclosure;

FIG. 5A illustrates effect of using a first traditional approach to PPG signal processing as applied to determining a hemoglobin concentration of FIG. 4A according to an embodiment of the disclosure;

FIG. 5B illustrates effect of using a second traditional approach to PPG signal processing as applied to determining a hemoglobin concentration of FIG. 4B according to an embodiment of the disclosure;

FIGS. 6A and 6B illustrate a block diagram and a schematic representation of a wearable device with function of determining a hemoglobin concentration according to various embodiments of the disclosure;

FIG. 7 illustrates a flow chart of a method for determining a hemoglobin concentration according to an embodiment of the disclosure;

FIG. 8 schematically illustrates an operation of transforming a PPG signal from a time to frequency domain according to an embodiment of the disclosure;

FIG. 9 illustrates key operations of a method for determining a hemoglobin concentration according to an embodiment of the disclosure;

FIG. 10 illustrates a key point of a transformation operation of FIG. 7 according to an embodiment of the disclosure;

FIGS. 11A and 11B illustrate particulars of transforming a PPG signal into a frequency domain, and the PPG signal in a time domain under different absorption conditions according to various embodiments of the disclosure;

FIG. 12 illustrates particulars of selecting a reference PPG signal in a frequency domain according to an embodiment of the disclosure;

FIG. 13 illustrates particulars of analyzing a reference PPG signal in a frequency domain according to an embodiment of the disclosure;

FIG. 14 illustrates a principle of compiling a database according to an embodiment of the disclosure;

FIG. 15 illustrates a final operation of a method for determining a hemoglobin concentration according to an embodiment of the disclosure; and

FIG. 16 illustrates results of predicting (determining) a hemoglobin concentration in a user's blood of a wearable device from a database according to an embodiment of the disclosure.

Throughout the drawings, it should be noted that like reference numbers are used to depict the same or similar elements, features, and structures.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

The proposed disclosure is a wearable device with function of determining a hemoglobin concentration in a user's blood, the device including at least one PPG sensor configured to irradiate a user's tissue, including skin, bones, blood, blood vessels, with radiation of at least two different wavelengths and to detect at least two PPG signals at the at least two different wavelengths in a reflection mode, wherein the wearable device is configured to transform the at least two PPG signals from a time domain to a frequency domain or a time-frequency domain, to describe the transformed PPG signals with respect to harmonics to obtain a set of characteristics, to determine a hemoglobin concentration in the user's blood based on the obtained set of characteristics and an information from a database.

According to an embodiment of the disclosure, the wearable device may further include a housing, a processor, a battery, and a memory accommodated within the housing.

According to an embodiment of the disclosure, the wearable device is configured to describe the PPG signals, which description includes selecting the most significant signal from all the PPG signals and using it as a reference signal; extracting harmonics from the reference signal and using them as coordinates; decomposing all the PPG signals according to said coordinates to obtain the set of characteristics; wherein the determination of a hemoglobin concentration includes comparing the obtained set of characteristics with the sets of characteristics from the database, each of which corresponds to a specific value of hemoglobin concentration.

According to an embodiment of the disclosure, the at least one PPG sensor of the wearable device includes at least one radiation source including two or more light-emitting diodes (LEDs).

According to an embodiment of the disclosure, the light-emitting diodes may be laser LEDs.

According to an embodiment of the disclosure, the at least one PPG sensor of the wearable device includes at least one radiation detector being preferably a wide-bandwidth photodetector or a photodiode.

According to an embodiment of the disclosure, one of said different wavelengths emitted by at least one PPG sensor of the wearable device is in the range of 495-570 nm, and preferably is 530 nm.

According to an embodiment of the disclosure, the PPG signals provided by the wearable device are back-reflected and/or back-scattered from the user's tissues.

According to an embodiment of the disclosure, the wearable device is further configured to filter out motion artifacts from the PPG signals. It should be noted that the analysis of a signal in a frequency domain makes an algorithm for determining a hemoglobin concentration to some extent resistant to motion artifacts, provided that the frequency and duration of these artifacts do not coincide with the main signal. The frequency of the artifacts may coincide with the frequency of the PPG signal, but the motion artifacts are non-stationary, i.e., their characteristics vary over time, so when the signal accumulates, it is possible to amplify the essentially stationary PPG components and to suppress the contribution of the motion artifacts to the overall signal spectrum.

According to an embodiment of the disclosure, the transformation of the PPG signals from the time domain to the frequency domain includes a Fourier transform or a Hilbert-Huang transform, and the transformation from the time domain to the time-frequency domain includes a wavelet transform.

According to an embodiment of the disclosure, the wearable device is configured to select the most significant signal characterized by the best signal-to-noise ratio, the highest amplitude and/or the least affected by noises.

According to an embodiment of the disclosure, the wearable device is configured to extract the harmonics from the reference signal, which extraction includes determining a coordinate of a fundamental harmonic and calculating coordinates of other harmonics.

According to an embodiment of the disclosure, the processor of the wearable device is configured to describe the PPG signals with respect to the harmonics, which description includes measuring, for each harmonic, an amplitude, an area under curve, a peak width at half height and/or a signal-to-noise ratio to obtain the set of amplitude-phase characteristics of the harmonics.

According to an embodiment of the disclosure, the wearable device is configured to perform the above-mentioned comparison of the obtained set of characteristics with sets of characteristics from the database using a numerical method or a prediction algorithm taking into account data from other users, the sets of characteristics from the database previously (in advance) measured from the other users and matched with hemoglobin concentrations previously measured by a laboratory method. Meanwhile, the prediction algorithm is acquired in the process of neural network training, wherein in the process of machine learning of neural network, the above sets of characteristics were used.

According to an embodiment of the disclosure, the memory of the wearable device is configured to store the database.

According to an embodiment of the disclosure, the wearable device is further configured to correlate the determined hemoglobin concentration using a hemoglobin concentration value measured by the laboratory method, update the database, and revise the prediction algorithm using a machine learning algorithm or a neural network.

According to an embodiment of the disclosure, the wearable device further includes an input and output device configured to input the user profile data into the memory and display the determined hemoglobin concentration, and a communication module configured to communicate with a remote server and/or a cloud storage.

According to an embodiment of the disclosure, the wearable device is further configured to determine an oxyhemoglobin concentration in a user's blood.

According to an embodiment of the disclosure, the wearable device is configured to be placed on a wrist or other parts of the body, for example, on the finger.

According to an embodiment of the disclosure, the wearable device is a smart device, such as a smartwatch or a fitness bracelet.

The proposed disclosure also relates to a method for determining a hemoglobin concentration in a user's blood, the method including irradiating a user's tissue with radiation of at least two different wavelengths; detecting PPG signals in a time domain at the at least two different wavelengths in a reflection mode, transforming the detected PPG signals from the time domain to a frequency domain or a time-frequency domain, describing the transformed PPG signals with respect to harmonics to obtain a set of characteristics, determining a hemoglobin concentration in the user's blood based on the obtained set of characteristics and an information from a database.

According to an embodiment of the disclosure, in the operation of describing the PPG signals, the most significant signal is selected from all the PPG signals and used as a reference signal, harmonics are extracted from the reference signal and used as coordinates, all the PPG signals are decomposed according to said coordinates, wherein, in the operation of determining a hemoglobin concentration, the obtained set of characteristics is compared with the sets of characteristics from the database, each of which corresponds to a specific value of hemoglobin concentration.

According to an embodiment of the method, the irradiation is performed by using at least one radiation source being two or more light-emitting diodes.

According to an embodiment of the disclosure, the irradiation is performed by using at least two laser light-emitting diodes.

According to an embodiment of the disclosure, the PPG signals are detected by using at least one radiation detector preferably being a wide-bandwidth photodetector or a photodiode.

According to an embodiment of the disclosure, one of the at least two different wavelengths of radiation to which the user's tissue is irradiated is in the range of 495-570 nm, and preferably is 530 nm.

According to an embodiment of the disclosure, the PPG signals are back-reflected and/or back-scattered from the user's tissues.

According to an embodiment of the disclosure, the method further including filtering out the PPG signals from motion artifacts. Filtering out the PPG signals from motion artifacts (body movement when walking, movement of hands, fingers, trembling, or the like) can be performed, for example, using data of an accelerometer of the wearable device, and is performed in order to further process the higher quality filtered signals.

According to an embodiment of the disclosure, the PPG signals are transformed from the time domain to the frequency domain by a Fourier transform or a Hilbert-Huang transform, and the PPG signals are transformed from the time domain to the time-frequency domain by a wavelet transform.

According to an embodiment of the disclosure, the most significant signal is characterized by the best signal-to-noise ratio, the highest amplitude and/or is the least affected by noises.

According to an embodiment of the disclosure, the extraction of the harmonics from the reference signal includes determining a coordinate of a fundamental harmonic and calculating coordinates of other harmonics.

According to an embodiment of the method, the description of the PPG signals with respect to the harmonics includes measuring, for each harmonic, an amplitude, an area under curve, a peak width at half height and/or a signal-to-noise ratio to obtain the set of amplitude-phase characteristics of the harmonics.

According to an embodiment of the disclosure, the comparison is performed by using a numerical method or a prediction algorithm taking into account profile data of other users, the sets of characteristics from the database previously measured from the other users, and hemoglobin concentrations previously measured by a laboratory method.

According to an embodiment of the disclosure, a machine learning algorithm or a neural network learning is used as the numerical method.

According to an embodiment of the disclosure, the database is configured to be stored on a memory, a remote server and/or in a cloud storage.

According to an embodiment of the disclosure, the method further includes correlating the determined hemoglobin concentration using the hemoglobin concentration value obtained by the laboratory method, updating the database, and revising the prediction algorithm using the machine learning algorithm or the neural network.

According to an embodiment of the disclosure, the method is further adapted to determine an oxyhemoglobin concentration in a user's blood.

The proposed disclosure also relates to a system for determining a hemoglobin concentration in a user's blood, the system including the above-mentioned wearable device and a remote server or a cloud storage, wherein the system includes a communication interface between the wearable device and the remote server and/or the cloud storage.

According to an embodiment of the disclosure, the database is adapted to be stored on the memory, the remote server and/or in the cloud storage and is configured to access the user data from external devices via the remote server and/or the cloud storage.

According to an embodiment of the disclosure, the user profile data and the results of previous hemoglobin concentration determinations are stored on the remote server and/or in the cloud storage.

According to an embodiment of the disclosure, the system further includes a remote server and/or a cloud storage accessing the user profile data and the results of previous hemoglobin concentration determinations from other electronic devices.

The proposed wearable device, method and system provide the possibility of non-invasive, continuous, reliable and motion resistant monitoring of blood properties, in particular the determination of a hemoglobin concentration and blood oxygen saturation, and are suitable for non-professional use.

Hereinafter, various embodiments of the disclosure will be described with reference to the drawings. Persons skilled in the art will be understood that various embodiments should in no way be construed as limiting the scope of the claimed disclosure, and that other tangible and technical means equivalent to or similar to those listed below may be used by skilled persons to perform various operations, functions, method steps, or the like, described below. The detailed description is not intended to limit the scope of the claimed disclosure, which is defined only by the appended claims.

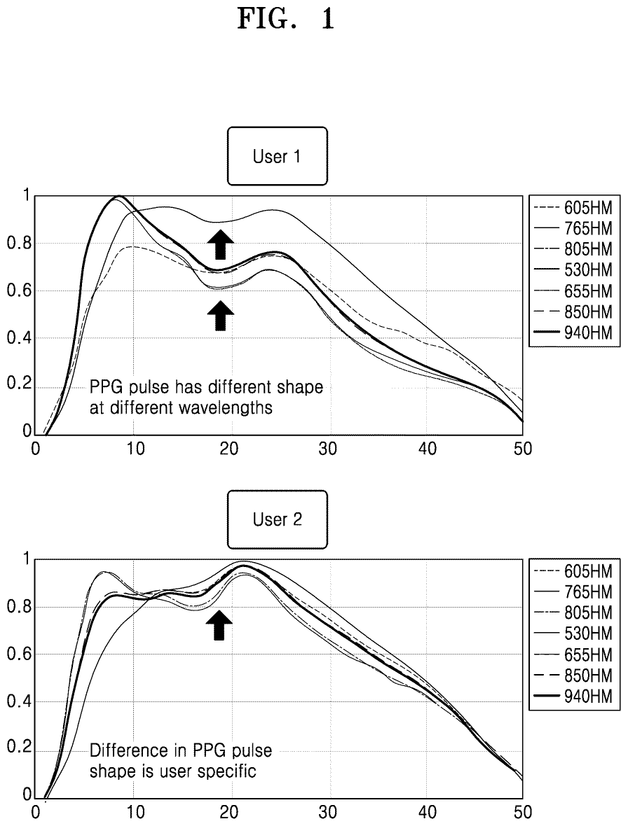

FIG. 1 schematically illustrates a dependence of a PPG signal shape on a wavelength and a user according to an embodiment of the disclosure.

Referring to FIG. 1, it can be clearly seen that the waveform of the pulse signal depends not only on a wavelength of primary radiation, but also on a specific user. The main factors influencing the shape of the PPG signal are absorption and scattering of radiation in a tissue, hydrodynamics of blood vessels, and mechanical deformation in the tissue. Meanwhile, a hemoglobin concentration in the patient's blood is determined, for example, by absorption of radiation in a tissue, and more specifically, as it will be discussed later, by a pulsating (AC) component of the absorption in arterial blood, and the contribution made to the signal due to scattering of radiation in the tissue, the hydrodynamics of blood vessels and the mechanical deformation is a source of errors and must be eliminated. Taking into account the particulars of processes of propagation of optical radiation in a biological tissue, that will be discussed further, the above processes (sources of errors) lead to a significant change in a shape of a pulse wave depending on a wavelength, and the waveform is specific in respect to the optical properties of the tissue of each individual user. Thus, for the correct measurement of a hemoglobin concentration in blood (determined by absorption of radiation in the arterial blood), a shape of the PPG signal is necessary to be analyzed. The important points here are the following: the need for spectroscopic measurements at multiple wavelengths, the need to take into account that at some wavelengths the PPG signal may be very weak, the signal may have a difficult shape to process, the need to measure a weak PPG signal having a complex shape.

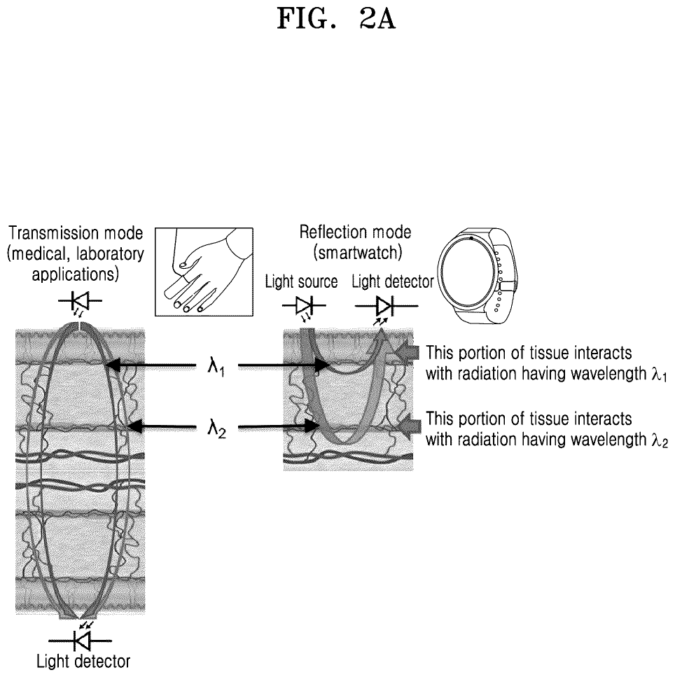

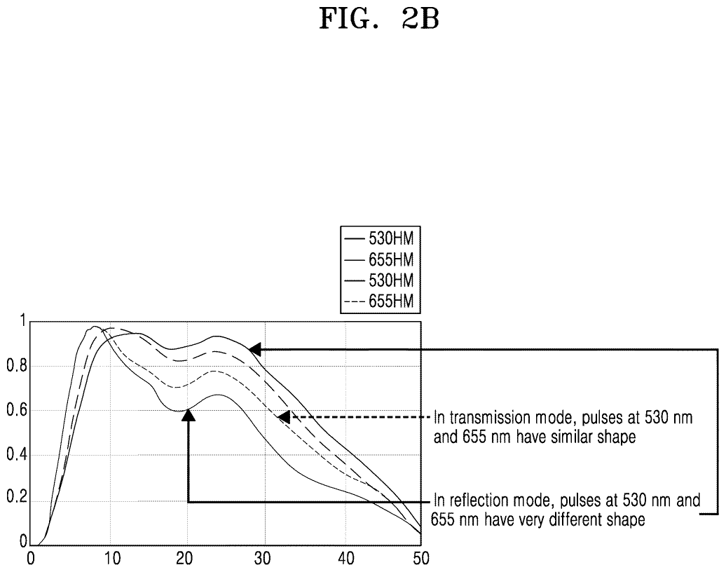

FIG. 2A schematically illustrates a difference in mechanisms of scattering and absorption of radiation in a tested tissue volume in a transmission mode and in a reflection mode according to an embodiment of the disclosure.

Referring to FIG. 2A, in the transmission mode used in medical and laboratory applications, radiation at two different wavelengths λ₁ and λ₂ travels approximately the same optical path through the patient's tissue from a light source to a light detector and interacts with the same tissue portions. In the reflection mode used in wearable devices, for example, in smartwatches and fitness bracelets, radiation at two different wavelengths λ₁ and λ₂ travels a different path from a light source to a light detector and interacts with different portions of the patient's tissue. Radiation with a wavelength λ₁ interacts, for example, only with a patient's skin and partially with muscle tissue, including capillary blood vessels, whereas radiation with a wavelength of λ₂ interacts with a patient's skin, with muscle tissue, including not only capillary blood vessels, but also venous and arterial vessels. However, for the correct measurement of hemoglobin, the same tested (probe) volume of tissue is required, which is difficult to provide in the reflection mode. Hence, it is necessary to find a solution to overcome this problem.

FIG. 2B schematically illustrates shapes of a PPG signal at wavelengths of 530 nm and 655 nm in a transmission mode and a reflection mode according to an embodiment of the disclosure.

Referring to FIG. 2A, the optical path of radiation at different wavelengths in the transmission mode is approximately the same, so the PPG signal in this mode has a similar shape at different wavelengths, for example, 530 nm and 655 nm, as shown by dashed lines in FIG. 2B. At the same time, the PPG signal in the reflection mode has a very different shape at different wavelengths, as shown by solid lines in FIG. 2B. In view of the foregoing, the main problems of spectral analysis of the PPG signal are the following:
AC component of the pulse signal has a complex shape (waveform), i.e., it is not a harmonic or sinusoidal signal;
a shape of AC component is radiation wavelength-dependent, i.e., the shape of the pulse signal carries an information, including about spectral properties of tissue;
limitedness of operation in the reflection mode.

When looking for a solution, it is important to take into account significantly different optical paths for different wavelengths, which is less important in the transmission mode and crucial for the reflection mode (for example, for smartwatches), the need to measure the same tissue portion, the different pulse shape caused by the difference in scattering and absorption of radiation. Thus, for the reflection mode, the shape of the PPG signal is preferably to be analyzed.

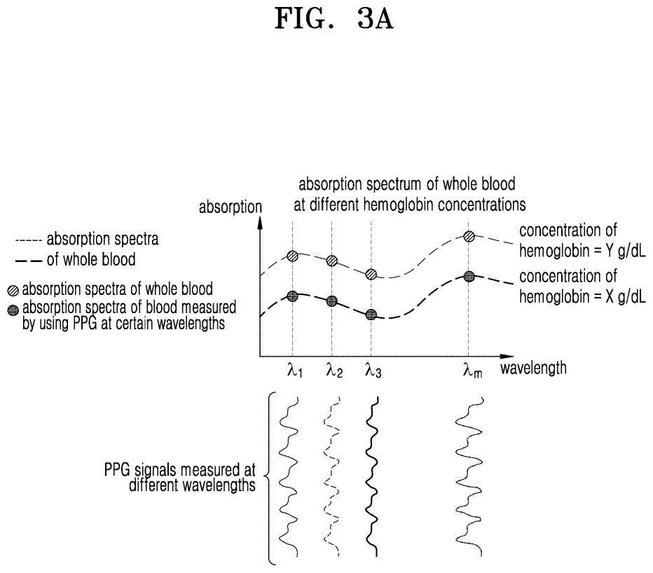

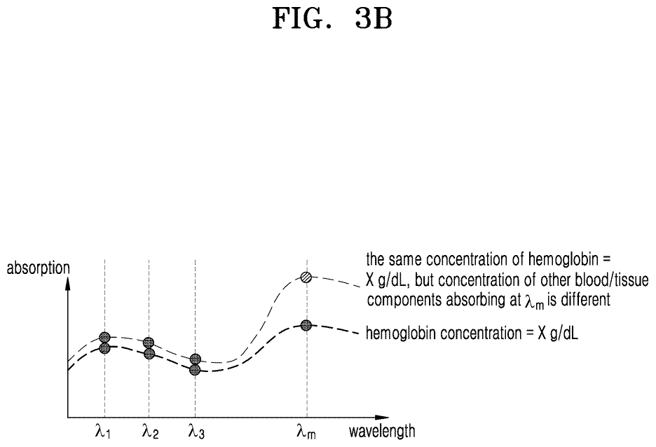

FIG. 3A schematically illustrates absorption spectra of whole blood at different hemoglobin concentrations, obtained by using photoplethysmography (PPG) at some radiation wavelengths according to an embodiment of the disclosure.

Referring to FIG. 3A, continuous absorption spectra of whole blood at two different concentrations of hemoglobin are shown by dashed lines, and absorption measured at several discrete points is shown by circles. It is apparent from FIG. 3A that under ideal conditions, when there is whole blood in a cuvette, a hemoglobin concentration can be determined by measuring the transmission of optical radiation at any one wavelength. However, the case of whole blood is ideal, and in real cases, measurements on whole blood cannot be performed in non-invasive manner.

FIG. 3B schematically illustrates absorption spectra at a same hemoglobin concentration, but in a presence in one case of other blood components or other tissues, including melanin, lipids, proteins, or the like according to an embodiment of the disclosure.

Referring to FIG. 3B, at some wavelengths, for example, at λₘ, a remarkable absorption of optical radiation occurs due to the presence of other blood components or other tissues. Thus, the other blood components or the other tissues can absorb at the same wavelengths as hemoglobin does. Hence, it is impossible to say definitively whether the absorption at the wavelength λₘ has changed because of hemoglobin or something else.

Referring to FIGS. 3A and 3B, the ideal case in blood analysis is to measure absorption of blood only, when the absorption at any wavelength corresponds to changes in a hemoglobin concentration of blood. In the real case, in practice, the absorption of whole tissue, including blood, is measured, wherein other components can also absorb radiation at the same wavelengths, affecting the pulse signal. Hence, the measurement of absorption at a single wavelength can lead to an incorrect result. Therefore, in this case, a spectroscopic approach to measurements is required, i.e., in order to correctly determine a hemoglobin concentration, the absorption spectrum it necessary to be measured at multiple wavelengths.

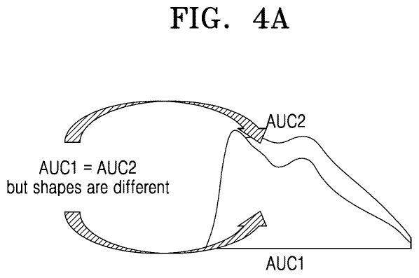

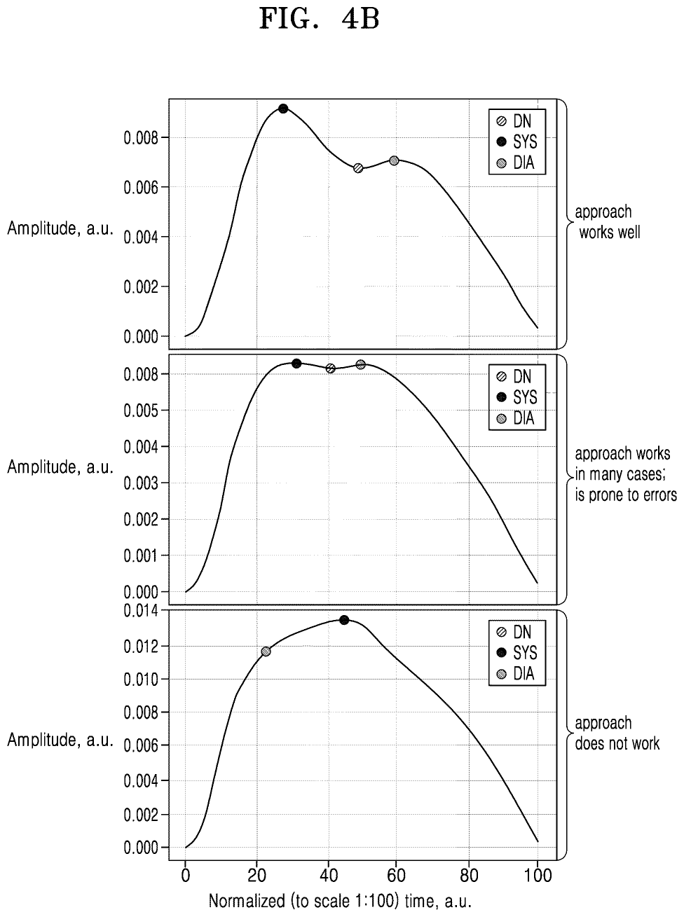

FIG. 4A schematically illustrates a first traditional approach to PPG signal processing, which consists in measuring an area under curve (AUC) of AC component of the PPG signal, i.e., determining an integral area of a shape of the PPG signal according to an embodiment of the disclosure.

Referring to FIG. 4A, it is believed that the intensity of the AC component thus measured is proportional to optical transmission of a measured tissue volume and can be recalculated into absorption at this portion according to the Bouguer-Lambert-Behr law. This is the simplest measurement approach applied in the transmission mode, as discussed above. Obviously, the signals depicted at FIG. 4A have approximately the same integral area, but different curve shapes. Since this approach does not take into account the information about the pulse shape, the measurement of optical transmission and absorption in the reflection mode is performed with a large error.

FIG. 4B schematically illustrates a second traditional approach to PPG signal processing, which consists in estimating a curve shape of a PPG signal according to an embodiment of the disclosure.

Referring to FIG. 4B, a first peak of a pulse wave, corresponding to the anacrotic period of the pulse wave, is formed during the systole period (“systolic peak” is designated as SIS). A peak value of the anacrotic phase is also referred to as a pulse wave amplitude and corresponds to a stroke volume of blood during cardiac output, thus providing indirect information about degree of inotropic effect. A second peak of the pulse wave, corresponding to the dicrotic period of the pulse wave, is formed by reflecting the volume of blood from the aorta and large great vessels and partially corresponds to the diastolic period of the heart cycle (“diastolic peak” is designated as DIA). The dicrotic phase provides an information about vascular tone. The top of the pulse wave corresponds to the largest volume of blood, and its opposite part corresponds to the smallest volume of blood in the tested portion of tissue. On the descending front of each wave, a depression is noticeable, i.e., a dicrotic incisure (notch), which corresponds to the closure of the aortic valve (“dicrotic notch” is designated as DN). The nature of the pulse wave depends on elasticity of a vascular wall, a pulse rate, a volume of a tested tissue, a width of lumen of vessels, or the like. It is believed that a frequency and duration of the pulse wave depends on particulars of heart performance, and the size and shape of its peaks depends on state of a vascular wall. If the systolic and diastolic peaks, as well as the depression therebetween are clearly expressed on the pulse wave (the upper PPG signal at FIG. 4B, rare case of an ideal curve specific for young, healthy people with a strong cardiovascular system), the approach to determining the optical properties of tissue by the curve shape of the PPG signal works well. In most cases, said particulars of the pulse wave are not clearly expressed (middle graph in FIG. 4B, specific for adults of relatively healthy people), this approach works in many cases, but is prone to errors, since it requires individual tuning of algorithm for each curve, and therefore, it is difficult to optimize processing. However, in some patients, the systolic and diastolic peaks, as well as the depression therebetween may be difficult to be determined or even indistinguishable (lower graph in FIG. 4B, rare case of a bad curve specific for elderly people with a weak cardiovascular system or for young unhealthy people). In this case, the curve shape of the waveform is characterized by the same parameters that have a clear physiological meaning, as described above, but the formalization of the algorithm for finding these points is difficult and therefore the shape is characterized inaccurately.

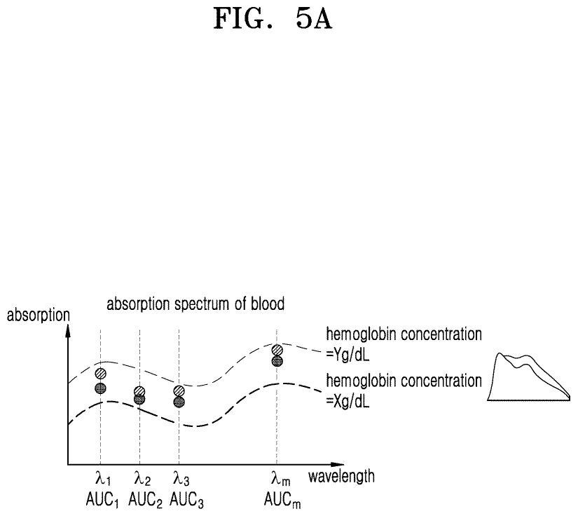

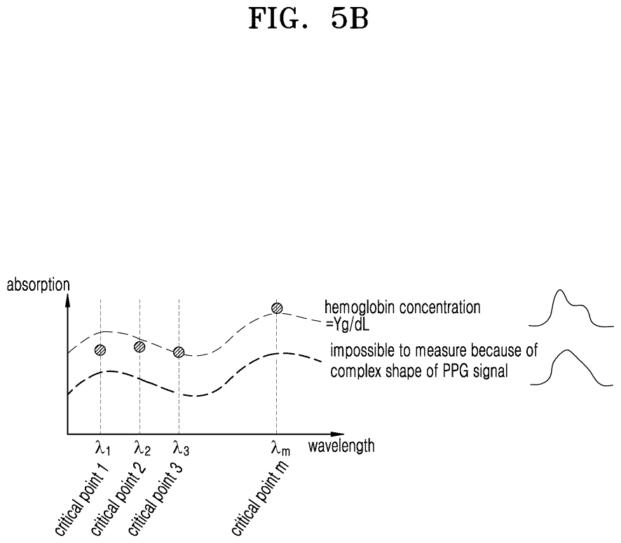

FIG. 5A further illustrates effect of using a first traditional approach (AUC determination) to processing the PPG signal of FIG. 4A on example of determining a hemoglobin concentration according to an embodiment of the disclosure.

Referring to FIG. 5A, absorption spectra of whole blood with different hemoglobin concentrations X and Y, i.e., dependencies of absorption on a wavelength of radiation are shown by dashed curves, absorption values determined by the areas of the PPG signal, i.e., by the integral areas of AC components of the signal are depicted by circles. Meanwhile, the circles for different concentrations in the depicted graphs are located close to each other and are markedly offset from true absorption values. This means that the determination of a hemoglobin concentration by the areas of AC component of the PPG signal results in an incorrect concentration value. Moreover, with this approach, it is often impossible to distinguish between two different concentrations of hemoglobin in blood.

FIG. 5B illustrates effect of using a second traditional approach to PPG signal processing as applied to determining a hemoglobin concentration of FIG. 4B according to an embodiment of the disclosure. In FIG. 5B, the term “critical point” means a set of critical points for a waveform curve at each of said wavelengths.

Referring to FIG. 5B, as for the use of the second traditional approach to processing the PPG signal as applied to the determination of a hemoglobin concentration by using critical points of the pulse signal, as shown in FIG. 5B, in one case, at the hemoglobin concentration Y (upper curve), the approach allows for performing enough accurate measurements, however, in other cases (lower curve) it is impossible to determine the absorption of radiation at some wavelengths at all due to the impossibility to distinguish critical points on the curve of the PPG signal, and therefore a hemoglobin concentration is impossible to be determined by the curve shape of the pulse signal.

In contrast to the approaches considered, the method according to the disclosure can be applied to any pulse signal shape, as will be described below.

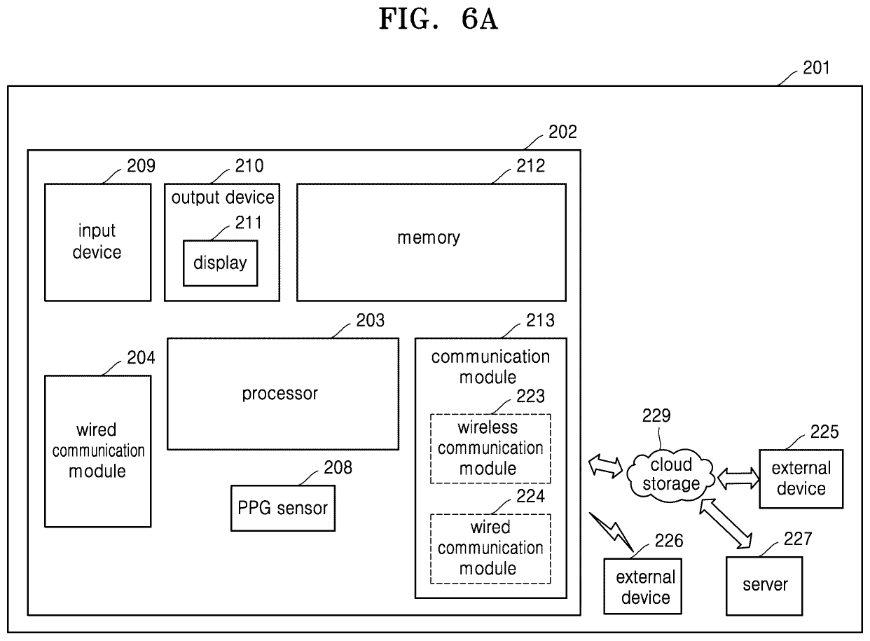

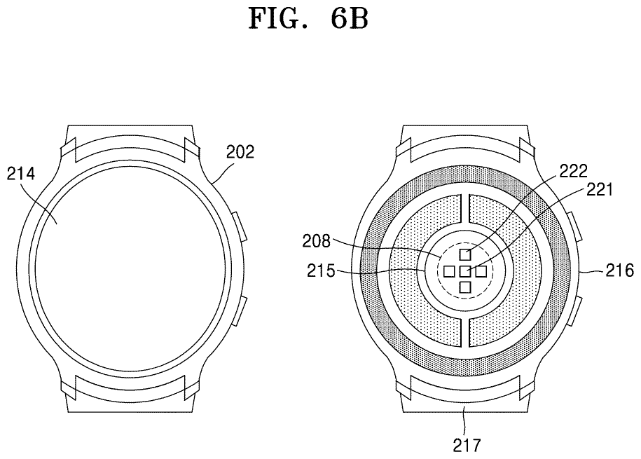

Hereinafter, a schematic representation of a wearable device 201 with function of determining a hemoglobin concentration in a user's blood will be illustrated with reference to FIGS. 6A and 6B.

FIGS. 6A and 6B illustrate a block diagram and a schematic representation of a wearable device with function of determining a hemoglobin concentration according to various embodiments of the disclosure.

Referring to FIGS. 6A and 6B, a block diagram of a preferred embodiment of the wearable device 201 comprising a housing 202, a processor (microprocessor) 203, a battery 204, at least one PPG sensor 208, an input device 209, an output device 210, a display 211, a memory 212, and a communication module 213 including a wireless communication module 223 and a wired communication module 224 will be described with reference to FIG. 6A. The input device 209 and the output device 210 may be configured as a single input and output device. The wearable device 201 may comprise additional sensors, for example, inertial movement sensors, gyroscopes, accelerometers, an electrocardiogram (EKG) measuring sensor, an atmospheric pressure sensor, a humidity sensor, a Hall sensor, an ambient illuminance sensor, or the like. In embodiments, the wearable device 201 may be a smart device, in particular a smartwatch or a fitness bracelet, a medical wearable health monitoring device with function of determining a hemoglobin concentrations, or the like. The wearable device is an electronic wearable device and can be configured to establish a wired or wireless communication channel with external devices, for example, devices 225, 226, such as smartphones, wearable fitness bracelets, voice assistants, smart televisions (TVs), smartwatches, and so on, or with a server 227, and can be configured to transmit data via a network 228 or a cloud storage 229.

The wearable device 201 with the PPG sensor 208, in a reflection mode, is capable of detecting the light back-scattered and/or back-reflected from user's tissues, including skin, bones, blood, blood vessels.

The wearable devices with a PPG sensor use a reflection mode to conveniently place the device, for example, on user's hand. The PPG sensor being worn on the user's hand or other body part is in contact with the skin. However, the PPG sensor, that measures in the reflection mode, may be affected by motion artifacts and pressure fluctuations. Any movement, for example, physical activity, can lead to motion artifacts that distort the PPG signal and limit the accuracy of physiological parameters measurement. Pressure fluctuations acting on the sensor, such as a contact force between the PPG sensor and a measurement site, i.e., the force with which the device is pressed against user's skin, can deform the geometry of artery due to compression. Thus, in the reflection mode, an amplitude of variable component of the PPG signal can be affected by the pressure exerted on the skin. Such influences on measurement of the PPG signal are described in the article Toshiyo Tamura, Current Progress of Photoplethysmography and SpO₂ for Health Monitoring, Biomedical Engineering Letters (2019) 9, pp. 21-36, (https://doi.org/10.1007/s13534-019-00097-w).

An electrical circuit of the PPG sensor may contain an amplifier, a high-pass filter (about 0.1 Hz) to cut off the constant component and obtain pulsating changes of a signal, a low-pass filter (about 30 Hz) to eliminate high-frequency noise, as well as a microprocessor. The used frequency range depends on the circuit design. The PPG sensor may have a wireless module for transmitting data to an external device. In a preferred embodiment of the disclosure illustrated in FIG. 6B, the wearable device 201 is configured in the form of a smartwatch that is adapted to be placed on a user's wrist. In other embodiments of the disclosure, the wearable device 201 may be configured to be placed on other parts of user's body, for example, on a hand finger.

Referring to FIG. 6B, the wearable device 201 is illustrated in a top view and a bottom view. According to this embodiment of the disclosure, the housing 202 of the wearable device 201 includes a first surface 214 (or a front surface), a second surface (or a rear surface) 215 and a side surface 216 surrounding a space between the first surface 214 and the rear surface 215, and attachment elements 217 for detachably attaching the wearable device 201 to a wrist for example with a watch strap.

According to an embodiment of the disclosure, the processor 203 is configured to receive the at least two PPG signals from the at least one PPG sensor 208, store them in the memory 212 and load them from the memory 212 to perform processing operations.

The processor 203 is configured to filter out the at least two PPG signals with different wavelengths from motion artifacts, separate the at least two PPG signals with different wavelengths into AC and DC components of time series, and transform these time series (a signal in a time domain) into a frequency domain or a time-frequency domain, followed by extracting their amplitude-phase characteristics of harmonics, wherein the transformation of said time series into the frequency domain may comprise a Fourier transform or a Hilbert-Huang transform, and the transformation of said time series into the frequency-time domain may comprise a wavelet transform or a bilinear frequency-time distribution.

The processor 203 is configured to receive additional data (for example, data from said additional sensors of the wearable device 201), store it in the memory 212 of the wearable device 201, and also load it from the memory 212 to perform operations, if necessary.

The processor 203 of the wearable device 201 is configured to transmit an information about a hemoglobin concentration in the user's blood determined based on readings of the sensors 208 to the output device 210 to inform the user.

The processor 203 is configured to load data received by using the communication module 213 into the memory 212 and/or load data from the memory 212 into the communication module 213 for transmitting them to an external device (for example, devices 225, 226, server 227 or cloud storage 229).

The battery 204 is configured to supply a power to at least one component of the wearable device 201.

It is illustrated with reference to FIG. 6B that the at least one PPG sensor of the wearable device 201 is located on its rear surface. Thus, during operation, the radiation surface of the PPG sensor is in contact with a user's wrist. Multiple PPG sensors may be provided for in the wearable device 201.

The at least one PPG sensor 208 is configured to measure at least two PPG signals with different wavelengths. In an embodiment of the disclosure shown in FIG. 6B, the at least one PPG sensor 208 is disposed on the side of the rear surface 215 of the housing 202 (the surface facing the user's skin). In one embodiment of the disclosure (not shown), the wearable device 201 may comprise two PPG sensors 208, each comprising one radiation source 221 and one radiation detector (receiver) 222, wherein the radiation sources 221 of these two PPG sensors emit a light of different wavelengths. In this embodiment of the disclosure, the two PPG sensors 208 are capable of capturing two PPG signals at different wavelengths. In one embodiment of the disclosure shown in FIG. 6B, the wearable device 201 comprises one PPG sensors 208 including two radiation sources 221 and two radiation detectors 222, wherein these radiation sources 221 emit a light of different wavelengths. In this embodiment of the disclosure, the one PPG sensor 208 is capable of capturing two PPG signals of different wavelengths. In an embodiment of the disclosure, the radiation source 221 may comprise a light-emitting diode (LED). Light barriers may be provided between the radiation sources and/or the radiation detectors to reduce noises and avoid cross-talks.

The input device 209 is configured to receive data that can be used by another component (for example, the processor 203) of the wearable device 201, input from outside (for example, by a user of the wearable device 201). In one of embodiments of the disclosure, the input device 209 may be configured to input a user profile. The processor 203 of the wearable device 201 may receive the user profile from the input device 209, store the user profile in the memory 212, read it from the memory 212, and use it, if necessary. Data voice input and output can also be provided for in the wearable device 201.

The user profile data may include gender, age, height, weight of the user. The input and output device may also comprise a screen for displaying an information, for example, a determined concentration of hemoglobin. The screen may be configured to use an on-screen keyboard for data inputting.

The output device 210 may be configured to output an information to a user on the first surface 214 of the wearable device 201. The output device 210 may include a display 211, which may be configured to display an output information and is located on the first surface 214 of the wearable device 201. Further, the display 211 may be configured to use the on-screen keyboard to input data, for example, a user profile.

The memory 212 in a preferred embodiment may be configured to store:
a database with sets of amplitude-phase characteristics of harmonics matched with hemoglobin concentrations; and
user profile data;
measurement data sets containing amplitude-phase characteristics of harmonics and including:
at least two PPG signals with different wavelengths measured by the at least one PPG sensor 208;
dates on which each measurement data set was collected;
times on which each measurement data set was collected; and
hemoglobin concentrations determined by means of the processor for each measurement data set, or the like.

The memory 212 can also store various additional data, for example, data captured from the additional sensors of the wearable device 201. In addition, the memory 212 may store various instructions that, when executed on the processor 203, cause the processor 203 to control components of the wearable device 201 associated with the processor 203 and perform various data processing or calculations.

The communication module 213 may be used to transmit data from or to the wearable device. According to an embodiment of the disclosure, the communication module 213 may include a wireless communication module (for example, a cellular/mobile communication module, a short-range wireless communication module, or a global navigation satellite system (GNSS) communication module) or a wired communication module (for example, a local area network (LAN) communication module, or a power line communication (PLC) module). Thus, the communication module 213 can support establishing a wireless communication channel between the wearable device 201 and an external device (for example, a device 225, a device 226, a server 227 or a cloud storage 229) and performing communication via the established communication channel. The communication module 213 may include one or more communication processors that operate independently from the processor 203 and support direct (for example, wired) communication or wireless communication. The respective one of these communication modules may communicate with an external electronic device via a first network 228 (for example, a short-range communication network, such as Bluetooth™, wireless communication (Wi-Fi), or infrared data transmission (IrDA)) or a second network 228 (for example, a long-range communication network, such as a cellular/mobile network, an Internet, or a computer network, for example, a local area network (LAN) or a wide area network (WAN)). These different types of communication modules can be implemented as a single component (for example, a single chip) or can be multi-component (for example, a plurality of chips).

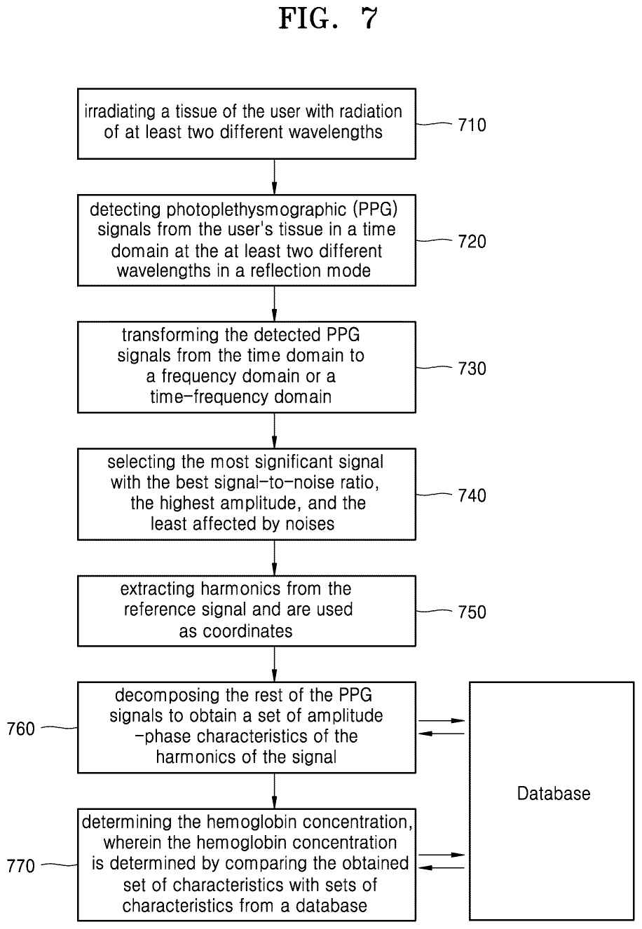

FIG. 7 illustrates a flowchart of a method for determining a hemoglobin concentration according to an embodiment of the disclosure. The claimed method can be performed by using the claimed wearable device with the function of determining a hemoglobin concentration in a user's blood.

Referring to FIG. 7, in operation 710, a user's tissue is irradiated with a light source emitting a light of at least two different wavelengths. In an embodiment, the wearable device irradiates a tissue of the user with radiation of at least two different wavelengths. In operation 720, PPG signals from the user's tissue are detected at the at least two different wavelengths. In an embodiment, the wearable device detects photoplethysmographic (PPG) signals from the user's tissue in a time domain at the at least two different wavelengths in a reflection. In operation 730, the detected PPG signals are transformed from a time domain to a frequency domain. In an embodiment, the wearable device transforms the detected PPG signals from the time domain to a frequency domain or a time-frequency domain. In operation 740, the most significant signal with the best signal-to-noise ratio, the highest amplitude, and the least affected by noises is selected from all the PPG signals available at the multiple wavelengths, and is used as a reference signal. In an embodiment, the wearable device selects the most significant signal with the best signal-to-noise ratio, the highest amplitude, and the least affected by noises. In operation 750, harmonics are extracted from the reference signal and are used as coordinates. In an embodiment, the wearable device extracts harmonics from the reference signal and are used as coordinates. In operation 760, the rest of the PPG signals are decomposed to obtain a set of amplitude-phase characteristics of the harmonics of the signals. In an embodiment, the wearable device decomposes the rest of the PPG signals to obtain a set of amplitude-phase characteristics of the harmonics of the signal. In operation 770, a hemoglobin concentration is determined, wherein the hemoglobin concentration is determined by comparing the obtained set of characteristics with sets of characteristics from a database, each of which corresponds to a specific value of hemoglobin concentration. In an embodiment, the wearable device determines the hemoglobin concentration. Meanwhile, the database is compiled in advance based on the obtained sets of characteristics for a plurality of users of the wearable device, wherein said sets of characteristics are correlated with specific hemoglobin values measured by a laboratory method.

Now the operations of the proposed method will be described.

A user's tissue is irradiated at the at least two wavelengths by using a light source, for example, LED(s). One of the at least two different wavelengths is in a range of 495-570 nm, i.e., the LED emits a green light, since it is usually the particularly green light provides the most informative signal in determining for example a hemoglobin concentration. A reflectometric method is used, i.e., the user's tissue is irradiated and then PPG signals from the user's tissue are detected at the at least two different wavelengths in a reflection mode.

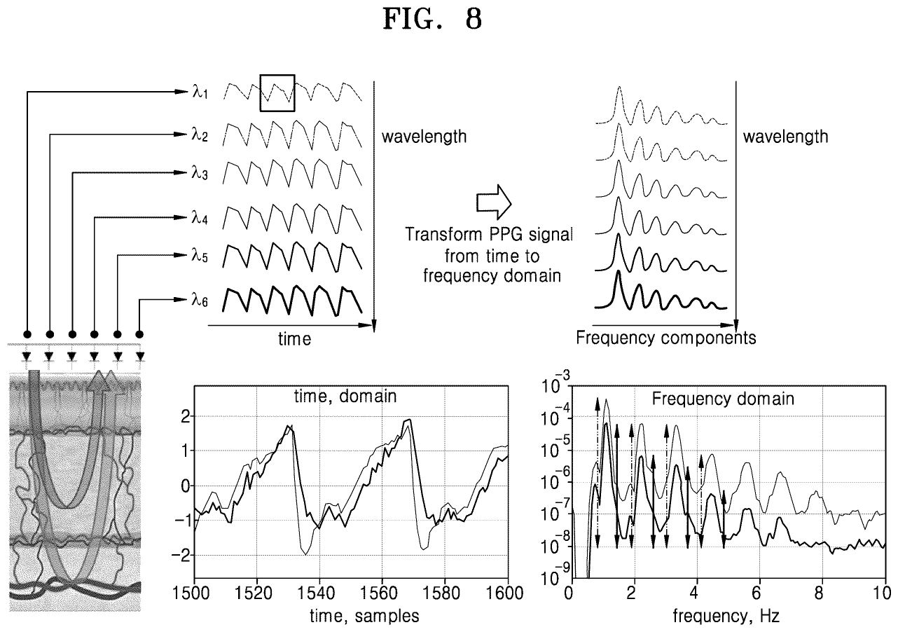

FIG. 8 schematically illustrates an operation of transforming a PPG signal from a time to frequency domain according to an embodiment of the disclosure.

Referring to FIG. 8, the signals are detected at six wavelengths. However, a number of the PPG signals at different wavelengths is not particularly limited. The detected signals are represented in a time domain (time series). In general, several representations of signal are possible: in a time domain, when f(t) is expressed as a function of time, in a frequency domain, when a spectrum (i.e., amplitudes of various harmonics) is determined, or in a frequency-time domain. Signals can be transformed from a time domain to a frequency/time-frequency domain by using various kinds of transformations. According to an embodiment of the disclosure, a Fourier transform serves as a means allowing for representing a detected signal with exponential components. However, the disclosure is not limited to the application of the Fourier transform and other transforms can be used, such as a Hilbert-Huang transform, a wavelet transform, a bilinear frequency-time distribution. The function F(ω) is the direct Fourier transform of the signal f(t). F(ω) represents the signal f(t) in the frequency domain. The time representation defines a certain signal at each moment in time, whereas the frequency representation characterizes relative amplitudes of frequency components of the signal, i.e., harmonics.

When performing the Fourier transform based on characteristics of the signal in the time domain, its amplitude-phase characteristics of harmonics (instantaneous spectrum, spectral density, or the like) can be obtained. The Fourier transform allows, for example, each PPG pulse to be transformed into a set of amplitudes and phases, and the thus obtained set of characteristics contains all the information necessary for a machine learning model used to determine a hemoglobin concentration. Meanwhile, for calculations, the set of signal characteristics is convenient to be described in complex numbers. Any of the above representations completely defines the signal. At the same time, as described in the “Description of Related Art” section, processing the PPG signal in the frequency domain allows for avoiding a number of prior art disadvantages.

The transformation of signals from the time domain to the frequency domain can also be performed by the Hilbert-Huang transform, and to the frequency-time domain by the wavelet transform or the bilinear frequency-time distribution.

The Hilbert-Huang transform (HHT) is a method of empirical mode decomposition (EMD) for nonlinear and non-stationary processes, i.e., decomposition into time-amplitude components, and subsequent Hilbert spectral analysis (HSA). The HHT is a time-frequency analysis of signals and does not require a priori functional basis of the transformation. The functions of the basis are obtained adaptively directly from the data by procedures for filtering out the functions of “empirical modes.” Instantaneous frequencies are calculated from the derived phase functions by a Hilbert transform of the functions of the basis. The main informative features of the Hilbert-Huang transform are an amplitude, an instantaneous frequency, and a phase of empirical modes.

The wavelet transform (WT) is an integral transform that is convolution of a wavelet function with a signal. The wavelet transform transfers a signal from a time representation to a frequency-time representation and is generalization of a spectral analysis, a typical representative of which is the classical Fourier transform. The term “wavelet” in English means “small (short) wave.” Wavelets are a generalized name for families of mathematical functions of a certain shape, which are local in time and frequency, and in which all functions are acquired from one basic (generating) function by means of its shifts and stretches along a time axis. Wavelet transform algorithms make it possible to take into account local changes in PPG signals and carry an information in a three-dimensional format, i.e., an amplitude, a frequency, a time. As a rule, wavelet transforms are divided into discrete (DWT) and continuous (CWT). DWT is used for signal transforming and encoding, CWT is used for signal analyzing. Presently, wavelet transforms are increasingly being used for a huge number of different applications, often replacing the usual Fourier transform.

Bilinear time-frequency distributions or quadratic time-frequency distributions arise in a subfield of signal analyzing and processing referred to as time-frequency signal processing, as well as in statistical analysis of time series data. Such techniques are used in cases where the frequency composition of a signal can vary for a time. Such an analysis was formerly referred to as time-frequency signal analysis, and is now more commonly referred to as time-frequency signal processing due to the progress in using these techniques to address a wide range of signal processing tasks. Compared to other frequency-time analysis techniques, such as for example the short time Fourier transform, the bilinear transform may not have greater clarity for most practical signals, but it provides an alternative basis for studying new definitions and new techniques. All the bilinear distributions are mutually convertible into each other.

At the bottom of FIG. 8, PPG signals at two different wavelengths in the time and frequency domains are illustrated.

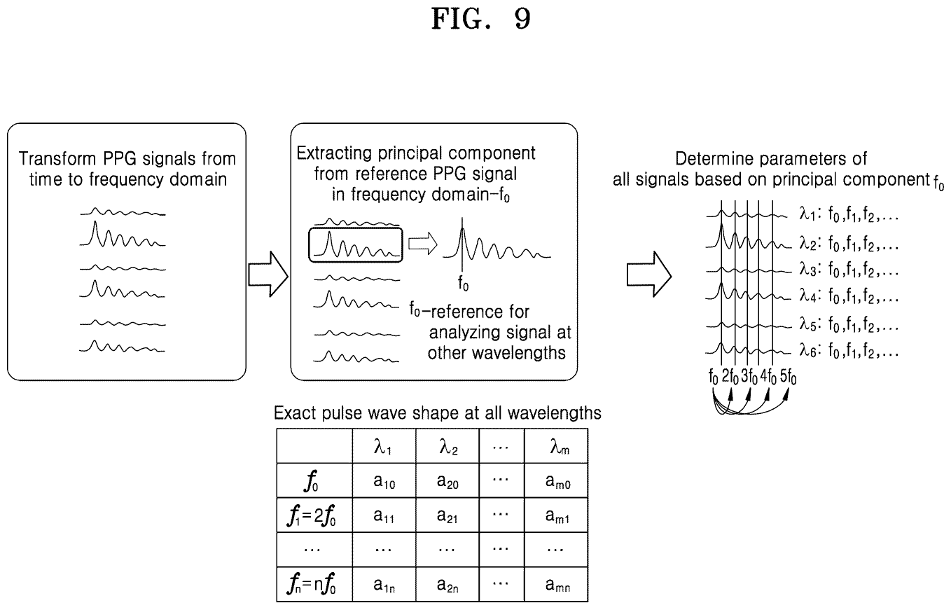

FIG. 9 illustrates key operations of a method for determining a hemoglobin concentration according to an embodiment of the disclosure.

Referring to FIG. 9, the disclosure allows for characterizing the exact pulse wave shape from the PPG signals, including weak and/or complex signals. Key operations of the method for determining a hemoglobin concentration illustrated in FIG. 9 are the following:
transforming the detected PPG signals from a time domain to a frequency domain or a time-frequency domain;
selecting, from all the detected PPG signals at different wavelengths in the frequency domain, the most significant signal that is characterized by the best signal-to-noise ratio, the highest amplitude and which is least affected by noises, and using the selected signal as a reference signal;
extracting a fundamental harmonic (principal component) from the reference signal and, based on it, other harmonics of the reference signal and using them as coordinates by which the rest of the PPG signals are decomposed, i.e., amplitude-phase characteristics of the harmonics are obtained.

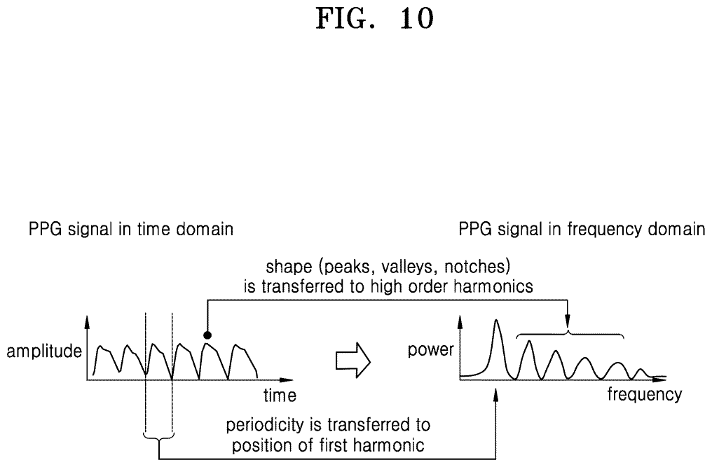

FIG. 10 illustrates a key point of a transformation operation of FIG. 7 according to an embodiment of the disclosure.

Referring to FIG. 10, key particular of the transformation operation of the method of FIG. 7 is illustrated in FIG. 10, which consists in that when transforming the PPG signal from the time domain to the frequency domain, an information about periodicity of the time signal is transferred to a position of a first harmonic of the frequency domain, and an information about the waveform (peaks, valleys, notches) is transferred to higher order harmonics. As the final result, data sets are obtained for each wavelength of the pulse signal in the frequency domain, each of which is characterized by specific characteristics (amplitudes a₁₀, a₁₁ . . . a₁ₙ at harmonic frequencies f₀, f₁ . . . fₙ) as shown at the bottom of FIG. 9. Meanwhile, a position, a number, an amplitude, a width of the harmonics obtained, which are easily measurable characteristics, totally describe the pulse wave in the frequency domain. Thus, the transformation of the PPG signals to the frequency domain makes it possible to extract the amplitude-phase characteristics of the harmonics of the signals.

Taking into account the above, processing the PPG signals in the frequency domain leads to the following advantages compared to processing in the time domain:
a finite number of characteristics (features) instead of continuous data of time series;
unique relationship between the characteristics and the PPG signal shape;
the presence of a new coordinate system (frequencies of harmonics) determined by one of the signals, which allows all signals to be uniformly decomposed according to a single coordinate system.

If the shape of the pulse wave is tried to be analyzed by the critical points, then each signal will have to be processed independently. In this case, a position of the critical points can “float,” thereby unambiguity in describing the signal shape at different wavelengths is violated. In addition, in case of weak signals and signals with a high noise level, finding the critical points may not be possible in principle. Due to the above-mentioned coordinate system (frequencies and positions thereof), characteristics (for example, amplitudes) of these frequencies can be found even in the signal spectrum with a high noise level.

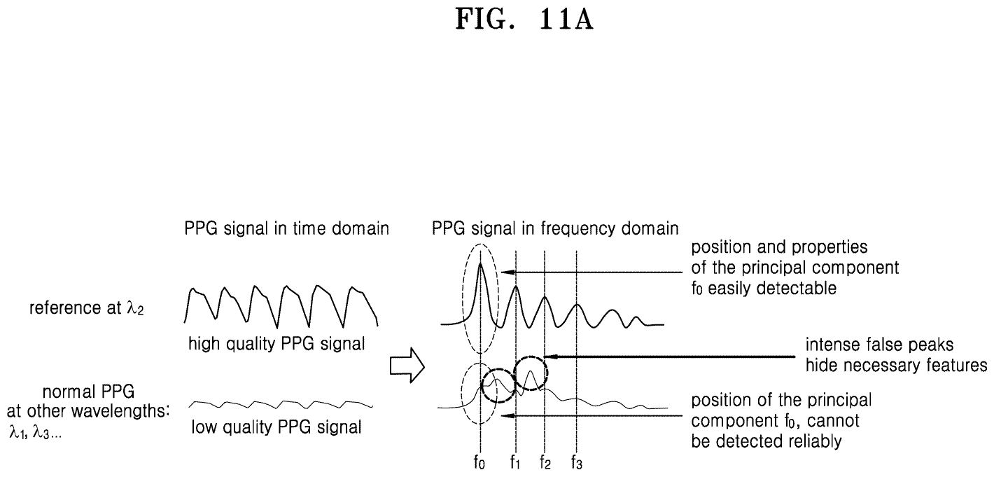

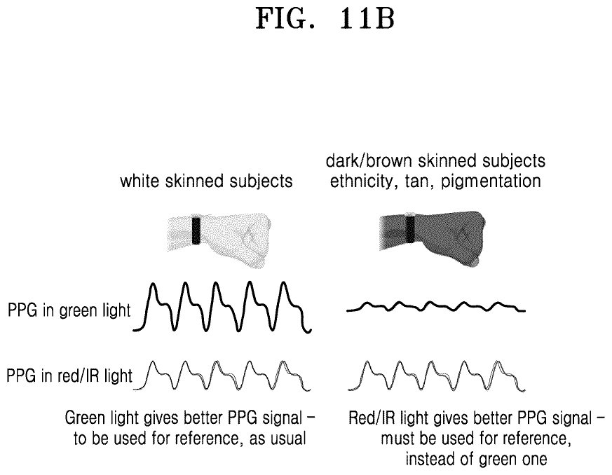

FIGS. 11A and 11B illustrate particulars of transforming a PPG signal into a frequency domain, and a PPG signal in a time domain under different absorption conditions according to various embodiments of the disclosure.

Referring to FIG. 11A, it illustrates particulars of transforming a PPG signal into a frequency domain. A high-quality reference PPG signal at wavelength λ2 and a normal low-quality PPG signal at other wavelengths λ₁, λ₃, or the like, are shown here. After the high-quality signal is transformed into the frequency domain, a position and characteristics of a fundamental harmonic f₀ can be easily measured. When similar transforming a low-quality signal, it is not possible to reliably determine a position of the fundamental harmonic f₀ in the frequency domain. Moreover, intense false peaks, which are shown in the graph on the right with broken circles, hide the necessary characteristics. However, an advantage of the disclosure is stability, or more accurately, independence of the reference PPG signal from interference and low-quality signals. After the Fourier transform is performed, the fundamental harmonic f₀ of the signal in the frequency domain is a first harmonic corresponding to the periodicity of the PPG signal, i.e., a heart rate. In case of other frequency-time transforms, the principal component is, for example, the frequency component of the highest power and the most stable in time with no direct relation to physiological parameters. Meanwhile, a position of the fundamental harmonic f₀ of a high-quality signal in the frequency domain corresponds to the position of the fundamental harmonic f₀ of low-quality signals.

FIG. 11B illustrates yet one particular of the PPG signal in the time domain, which affects the necessity to select an appropriate wavelength for reference measurements. According to the prior art, the best PPG signal is obtained when using a green light (˜530 nm). However, this is not always the case. The pulse signal is highly dependent on a person's skin color, his(her) ethnicity, tan, skin pigmentation, or the like. FIG. 11B shows PPG signals for white-skinned and dark-skinned people. The selection of a higher quality PPG signal is generally based on determining the highest signal-to-noise ratio.

Referring to FIG. 11B, for a white-skinned person, the best PPG signal is actually obtained in a green light and used as a reference signal. However, dark-skinned people have their own characteristics, the main of which is associated with that a green light penetrates to a shallow depth into patient's tissues. Since a green light is almost totally being absorbed by melanin in a dark skin, it does not reach blood vessels. At the same time, a red/IR light penetrates deeper into the tissues because melanin absorbs the red/IR light much less compared to a green light and does not affect the PPG signal. Hence, for the dark-skinned people, the best result will be obtained by using, as a reference signal, a PPG signal obtained by using a red/infrared light, rather than a green light, as for the light-skinned people.

Thus, individual settings are necessary, and an appropriate reference wavelength is desirable to be determined for each user of the wearable device individually.

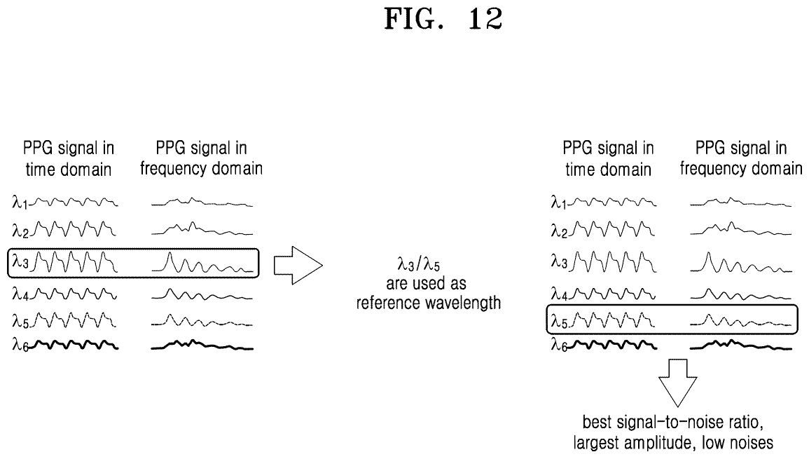

FIG. 12 illustrates as an example two sets of PPG signals in time and frequency domains at different wavelengths, wherein in one case, a signal obtained by using a green light for example with a wavelength λ₃, and in another case, a signal obtained by using a red light for example with a wavelength λ₅ is selected as a reference signal with the best signal-to-noise ratio, the highest amplitude and low noises according to an embodiment of the disclosure.

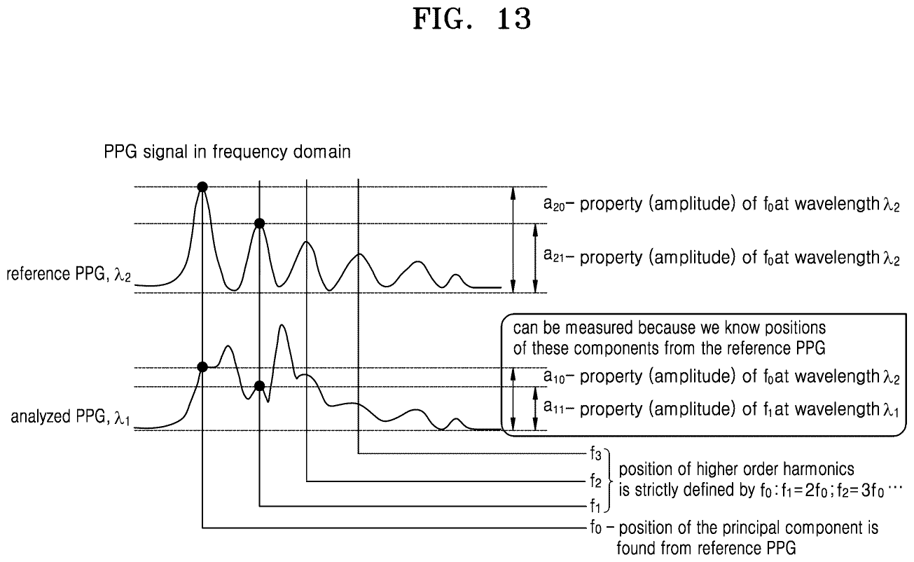

FIG. 13 further illustrates particulars of analysis of a signal in a frequency domain. A signal with a wavelength λ₂ was selected as a reference PPG signal according to an embodiment of the disclosure.

Referring to FIG. 13, in the top graph of FIG. 13, characteristics of the first f₀ and second f₁ harmonics of the PPG signal at the wavelength λ₂, namely the amplitudes a₂₀ and a₂₁ of these harmonics are shown. It should be again noted that in the frequency domain, the positions of harmonics are identical for signals of all wavelengths of a visible radiation spectrum. In other words, the positions of harmonics of the analyzed PPG signal with wavelength A shown in the lower graph coincide with the positions of harmonics of the reference PPG signal with wavelength λ₂. Hence, PPG signals at all wavelengths can be analyzed identically and simultaneously once harmonics are determined from the reference PPG signal. Meanwhile, the positions of higher-order harmonics are uniquely related to the positions of the fundamental harmonic by the following relations: f₁=2f₀; f₂=3f₀, or the like. Thus, having known the positions of harmonics of the reference signal, the positions, and therefore the amplitudes of the corresponding harmonics of the analyzed PPG signal with a wavelength λ₁, for example, amplitudes a₁₀ and a₁₁ of the first f₀ and the second f₁ harmonics can be easy determined.

According to the above technique, characteristics, in particular, amplitudes of all PPG signals at other wavelengths are determined based on the position of the fundamental harmonic f₀ of the reference PPG signal. The advantage of this technique is easy parameterization of a PPG signal shape simultaneously at all radiation wavelengths.

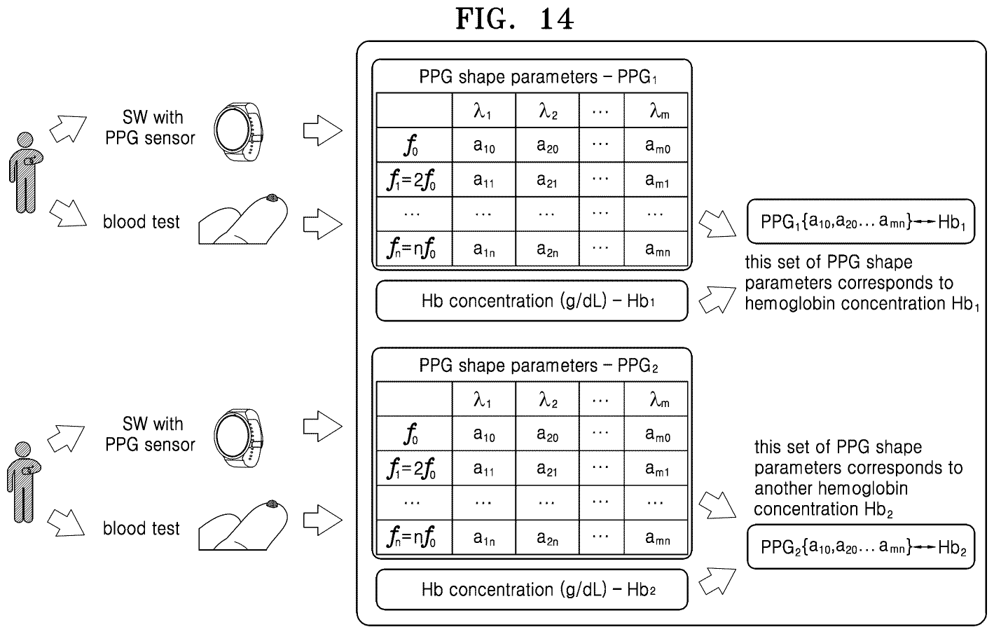

FIG. 14 illustrates a principle of compilation of a database for non-invasive, personal and/or on-demand monitoring of blood properties for example a hemoglobin concentration according to an embodiment of the disclosure.

Referring to FIG. 14, in the disclosure, this is provided as follows. As a result of machine learning of a neural network, a prediction algorithm is built in advance. In order to train the prediction algorithm that allows for determining (predicting) a hemoglobin concentration, a database was compiled. To this end, for each of a plurality of users of the wearable device, a set of amplitude-phase characteristics of harmonics of PPG signals in a frequency domain was determined (PPG:{a₁₀, a₂₀ . . . aₘₙ}, PPG₂{a₁₀, a₂₀ . . . aₘₙ}, or the like) by using a PPG sensor installed in the wearable device. In addition, for each of the plurality of users, a hemoglobin concentration (Hb₁, Hb₂, or the like) was determined by a reference method for example by performing blood analysis by any of laboratory methods. Thereafter, the corresponding sets of PPG signal characteristics determined by using the wearable device were correlated with the hemoglobin concentrations determined by using the laboratory blood analysis to obtain a database in which the set of characteristics PPG₁ corresponds to the hemoglobin concentration Hb₁, the set of characteristics PPG₂ corresponds to the hemoglobin concentration Hb₂, or the like. The sets of characteristics obtained by using the wearable device and the hemoglobin concentrations determined by the laboratory method were recorded in the database. Thus, the database contains all possible combinations of a hemoglobin concentration with the PPG signal characteristics, i.e., a plurality of data sets of the PPG signal, each of which is matched with a specific hemoglobin concentration. The thus compiled database was used for machine learning of the prediction algorithm. The prediction algorithm used in the processor of the wearable device provides the determination of a hemoglobin concentration based on the sets of characteristics obtained by processing the PPG signals for a specific user of the wearable device.

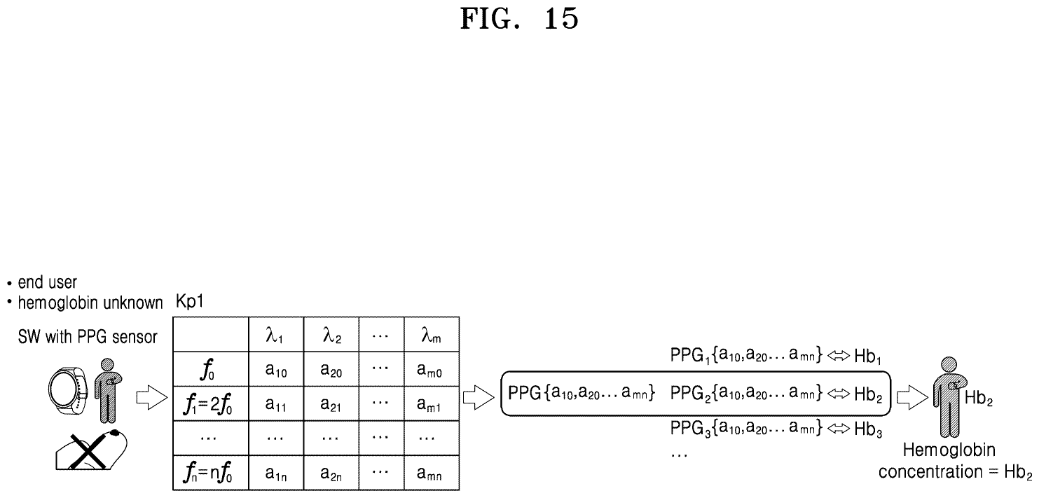

FIG. 15 illustrates a final operation of the non-invasive method for determining a hemoglobin concentration according to an embodiment of the disclosure.

Referring to FIG. 15, in this operation, in order to monitor a hemoglobin concentration in blood, a user uses the wearable device with the PPG sensor. First, a set of PPG signal characteristics for the user is measured by using the wearable device, then the database is accessed, and a set of PPG signal characteristics that are closest to the characteristics of the measured signal is determined therein by using the prediction algorithm or the numerical method. Since the characteristics of the measured signal and the signal determined from the database are essentially coincided, the hemoglobin concentration corresponding to the signal from the database will also correspond to the measured signal.

Referring to FIG. 15, the set of data of the measured PPG signal coincides with the data set of PPG₂, and therefore corresponds to the hemoglobin concentration Hb₂.

Thus, in order to determine a hemoglobin concentration by the claimed method:

1. PPG signals at multiple wavelengths are detected from a user with an unknown hemoglobin concentration and transformed them into a frequency domain.

2. Amplitude-phase characteristics of harmonics describing the PPG signals at multiple wavelengths are extracted: PPG {a₁₀, a₂₀ . . . aₘₙ}.

3. The obtained set of characteristics is compared with the sets of characteristics PPG₁, PPG₂ . . . PPGₙ from the compiled in advance database, as shown above.

4. A hemoglobin concentration is determined.

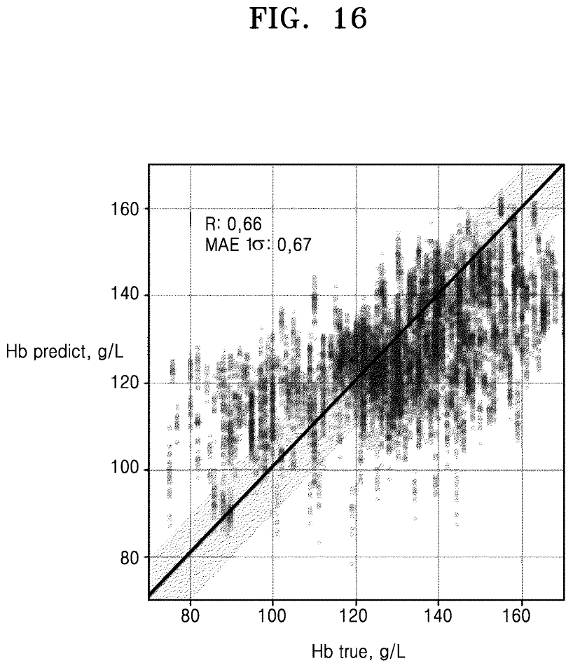

FIG. 16 illustrates a graph of results of predicting a hemoglobin concentration in blood of a user of a wearable device from a database containing an information on 840 tests performed on 170 tested subjects according to an embodiment of the disclosure.

Referring to FIG. 16, when collecting reference data, a laboratory analysis of capillary blood by a colorimetric method was used based on a hemoglobinazide assay, through which a hemoglobin concentration in blood of a tested subject was determined.

In the graph, a X-axis represents a hemoglobin concentration of a tested subject measured by the laboratory method, a Y-axis represents a hemoglobin concentration predicted by using the prediction algorithm. The X=Y line shows the correlation. In this case, a regression model of machine learning by the gradient descent method and k-fold cross-validation were used to evaluate the prediction performance.

Characteristics of accuracy of the prediction of the hemoglobin concentration by one of the models used are: MAE 1σ=0.67, R=0.66; where MAE (Mean Absolute Error) is a mean absolute error, which is a measure of errors between paired measurements of the same phenomenon, i.e., in this case, between the actual values of hemoglobin concentration measured by the laboratory method and the predicted values obtained by using the wearable device; MAE is calculated as a sum of absolute errors divided by the sample size; 1σ indicates that only those measurements whose error falls within one standard deviation from the mean error value at all points are taken into account; R is the Pearson correlation coefficient.

The obtained data clearly demonstrate that the hemoglobin concentration in blood of the tested subject can be predicted based on at least a set of measurement data read from the sensors of the wearable device, with high accuracy relative to the readings obtained by the laboratory method. As an example, in one of the prediction models, MAE 1σ is currently 0.67 g/L. Obviously, the prediction accuracy will increase both as the prediction algorithm is refined and as the information with the results of measurements on the tested subjects is accumulated in the database. Thus, it is expected that a hemoglobin concentration will be possible to be determined with high accuracy (within 1%) by using the wearable device according to the disclosure.

The solution of the problem of predicting a hemoglobin concentration in a user's blood was based on identifying empirical patterns in the training data placed in a common database by a machine method.

In the above embodiment of the disclosure, the prediction algorithm is built up for a wearable device located on a wrist because training of the prediction algorithm was based on data captured by sensors of the wearable device located on the wrist. In other embodiments of the disclosure, the prediction algorithm is possible to be trained taking into account the placement of the wearable device on other parts of the body, for example, on a finger. The prediction algorithm described herein can be implemented as software including one or more instructions that can be executed by the processor 203 of the wearable device 201 or by external processors/processing devices.

In the above process of training the prediction algorithm, profile data of other users (tested subjects), sets of measurement data measured in advance from the other users, and sets of reference data measured in advance by the laboratory method were used, as described above.

As discussed below, the database can be constantly updated, and in addition, the prediction algorithm is refined by machine learning.

In the process of machine learning, methods for selecting and processing of characteristics and corresponding discrete frequencies can be used:
in the Relief-F method, a vector of weights of features (characteristics) is calculated and normalized, and then the features whose weight exceeds the value of a predetermined threshold are selected;
the Correlation-based Feature Selection (CFS) method combines an evaluation formula with an appropriate correlation measure and a heuristic search strategy;
the Fast Correlation-based Filter method starts to operate with a full set of features, uses a measure of symmetric uncertainty to determine dependencies between the features and allows for selecting a subset by searching for and sequentially eliminating little informative features;
the Sequential Forward Feature Selection (SFFS) method at each iteration adds a feature to a set that provides the best recognition efficiency for this iteration;
the Mutual Information method determines a nonlinear correlation relationship instead of calculating the Pearson correlation “feature-feature” and “feature-tag”;
basic machine learning (artificial intelligence) algorithms; a combination of said regression methods and any derived regression methods, which are based on a basic algorithm, can be used as a working solution:
Decision trees/random forests;
the support vector machines method;
Linear analysis;
Deep learning methods—artificial neural networks.

### Best Embodiment

In order to implement the method according to the disclosure, for example, a smartwatch comprising a PPG sensor is used. The LED(s) built into the smartwatch emits a light at least two wavelengths, which is partially absorbed in tissues of a user's hand, and partially reflected and detected by the PPG sensor. The sensor detects the light at at least two wavelengths and a set of the detected PPG signals in a time domain is obtained. Then, using a processor built in the smartwatch, which is also used for subsequent operations, a reference PPG signal is selected at a certain wavelength. The reference PPG signal is selected among all available signals at different wavelengths based on the best signal-to-noise ratio, the highest amplitude, and minimum noises (most often this is a signal at a green light wavelength). Thereafter, the PPG signals are transformed from the time domain to a frequency domain using a Fourier transform. A position of a first harmonic (principal component) f₀ is extracted in the frequency domain from the reference PPG signal and used to analyze signals at other wavelengths. Based on the position of the first harmonic f₀, amplitude-phase characteristics of harmonics of all the signals are determined. Thus, for all combinations of wavelengths of the PPG signal and harmonics, a set of characteristics a₁₀, a₂₀ . . . aₘ₀, a₁₁, a₂₁ . . . aₘ₁ . . . a₁ₙ, a₂ₙ . . . aₘₙ is obtained, which uniquely describes the shape of the pulse wave at each wavelength λ.

### Further Embodiment 1

The embodiment relates to the use of a machine leaning (ML) method in the claimed method. As in the above embodiment of the disclosure, PPG signals at multiple wavelengths are firstly detected from a user with an unknown hemoglobin concentration in blood, then characteristics describing the PPG signals at the multiple wavelengths are extracted to obtain a set of characteristics: PPG {a₁₀, a₂₀ . . . aₘₙ}. In contrast to the above embodiment of the disclosure, a situation may occur where, when comparing the obtained set of characteristics with the sets included in the database, it will not be possible to unambiguously determine a hemoglobin concentration since the obtained set will not unambiguously correspond to any set from the database, being approximately between two sets that indicate of concentrations Hb₁ and Hb₂ respectively. In this case, a laboratory analysis of the user's blood is performed, an obtained result is included into the database, while correlating it with the obtained set of characteristics, and additionally the prediction algorithm is refined by using the machine learning method. In addition, the prediction algorithm can be trained by using machine learning to predict the association of existing sets of characteristics of the PPG signal shape with different concentrations of hemoglobin.

The advantageous effects of using the machine learning method are to detect blood parameters for a user whose data is not included in the database and to improve the accuracy of determining the blood parameters.

### Further Embodiment 2

The embodiment is aimed at determining a reference PPG signal more accurately. As described above, the reference signal is usually selected based on the best signal-to-noise ratio, the highest amplitude, and the signal having a green light wavelength is most commonly used. Thus, if a wavelength at which a good signal is obtained is known, additional hardware can be used together with the wearable device, which increases the intensity of the PPG signal at a predetermined wavelength, for example, a green light wavelength and determines it as a reference signal. After the reference signal is thus selected, further operations of the method are performed and a hemoglobin concentration is determined.

The advantageous effects of using this approach are that there is no need for an algorithm for searching a reference signal, and that the method works for any user (regardless of his physiological characteristics, skin color, or the like).

### Examples of Use of Disclosure

The disclosure can be used for non-intrusive monitoring a hemoglobin concentration of a user of the wearable device, such as a smartwatch or a fitness bracelet. When a hemoglobin concentration of a user is constantly monitored, both short-term and long-term deviations from the range of normal hemoglobin concentration can be detected. The short-term deviations are not critical and can be eliminated personally by the user based on available recommendations. For example, when the hemoglobin concentration is short-term decreased, which most often indicates anemia, it may be recommended to change the diet, increase physical activity, be more often in the fresh air, or the like. At the same time, the long-term deviations may indicate serious health problems and the need to visit a doctor. Meanwhile, the obtained data can be sent to a doctor remotely to gather initial anamnesis.

The advantage of such use of the disclosure is in low power consumption of the wearable devices, and therefore, a longer period of continuous monitoring, and better data analysis. In addition, a delicate interaction with a user is provided.

Another example of the use of the disclosure is use by athletes. As noted above, the main function of hemoglobin is the binding and transport of oxygen to internal organs. Deviation of a Hb concentration value from the norm leads to a decrease in endurance during sports activities. A low hemoglobin concentration leads to oxygen starvation of muscles (hypoxia or a low number of red blood cells), resulting in: weakness, periodic dizziness, fatigue, muscle pain, shortness of breath, cardiopalmus. A high hemoglobin concentration leads to dehydration or a high number of red blood cells, resulting in: weakness, nosebleeds, fatigue, muscle pain. Such states are difficult to tolerate, especially during physical activity. Muscles need oxygen to get enough energy, and scarcity of oxygen entails the inability of fibers to contract adequately. Athletes may not immediately notice the symptoms of the onset of anemia, which is associated with increased endurance of a body. Hence, the hemoglobin concentration is necessary to be known before training, and it is better to constantly monitor it.

Moreover, oxygen starvation is fraught with consequences for ordinary people since scarcity of oxygen in blood can adversely affect brain function.

Yet another example of the use of the disclosure is assistance in selecting a training regime, which is actually for novice athletes, athletes after a long break in training, triathletes, female athletes and high altitude training. An athlete using the smartwatch switches it to the hemoglobin concentration measurement mode. Taking into account the continuous sensor data, user profile and geolocation, the machine learning algorithm analyzes the data received from the hemoglobin sensor, which is then provided to the user in the form of a hemoglobin concentration indication. Based on the monitored hemoglobin concentration, personalized recommendations for training, such as regime, duration, amount of load, or the like, can be provided.

The disclosure extends the functionality of the Samsung Health app in smartwatches, while providing one more parameter for complex analysis of user's health. The smartwatch can provide the following indicators: daily variations in a hemoglobin concentration, a current hemoglobin concentration within the normal hemoglobin concentration, continuous measurement result during the day, average values for a predetermined period.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. A wearable device with a function of determining a hemoglobin concentration in a user's blood, the wearable device comprising:
at least one photoplethysmographic (PPG) sensor configured to irradiate a tissue of the user with radiation of at least two different wavelengths and to detect at least two PPG signals at the at least two different wavelengths in a reflection mode,
wherein the wearable device is configured to:
transform the at least two PPG signals from a time domain to a frequency domain or time-frequency domain,
describe the transformed PPG signals with respect to harmonics to obtain a set of characteristics, and
determine a hemoglobin concentration in the user's blood based on the obtained set of characteristics and an information from a database.

2. The wearable device of claim 1,
wherein the wearable device is configured to, when describing the PPG signals:
select the most significant signal from all the PPG signals and use it as a reference signal,
extract the harmonics from the reference signal and use the extracted harmonics as coordinates, and
decompose all the PPG signals according to the coordinates to obtain a set of amplitude-phase characteristics of the harmonics, and
wherein the wearable device is configured to, when determining a hemoglobin concentration, compare the obtained set of characteristics with sets of characteristics from the database, each of which corresponds to a specific value of hemoglobin concentration.

3. The wearable device of claim 1, wherein the at least one PPG sensor comprises at least one radiation source comprising two or more light-emitting diodes.

4. The wearable device of claim 1, wherein the at least one PPG sensor comprises at least one radiation detector, preferably being a wide-bandwidth photodetector.

5. The wearable device of claim 1, wherein one of the at least two different wavelengths is in a range of 495-570 nm, and preferably is 530 nm.

6. The wearable device of claim 1, wherein the wearable device is further configured to filter out motion artifacts from the PPG signals.

7. The wearable device of claim 1,
wherein the transformation of the PPG signals from the time domain to the frequency domain comprises a Fourier transform or a Hilbert-Huang transform, and
wherein the transformation from the time domain to the time-frequency domain comprises a wavelet transform.

8. The wearable device of claim 2, wherein the most significant signal is characterized by the best signal-to-noise ratio, the highest amplitude and/or the lowest noises.

9. The wearable device of claim 2, wherein the extraction of the harmonics from the reference signal comprises:
determining a coordinate of a fundamental harmonic; and
calculating coordinates of other harmonics.

10. The wearable device of claim 1, wherein description of the PPG signals with respect to harmonics comprises measuring, for each harmonic, an amplitude, an area under curve, a peak width at half height and/or a signal-to-noise ratio to obtain the set of amplitude-phase characteristics of the harmonics.

11. The wearable device of claim 2, wherein a comparison is based on a use of a numerical method or a prediction algorithm taking into account data from profiles of other users, the sets of characteristics from the database previously measured from the other users, and hemoglobin concentrations previously measured by a laboratory method.

12. The wearable device of claim 1, further comprising:
a memory configured to store the database.

13. The wearable device of claim 11, wherein the wearable device is further configured to:
correlate the determined hemoglobin concentration using a hemoglobin concentration value measured by the laboratory method;
update the database; and
revise the prediction algorithm using a machine learning algorithm or a neural network.

14. The wearable device of claim 1, further comprising:
an input and output device configured to input the user profile data and display the determined hemoglobin concentration; and
a communication module configured to communicate with a remote server and/or a cloud storage.

15. The wearable device of claim 1, wherein the wearable device is further configured to determine an oxyhemoglobin concentration in the user's blood.

16. The wearable device of claim 1, wherein the wearable device is configured to be placed on a wrist.

17. The wearable device of claim 1, wherein the wearable device is a smart device, preferably a smartwatch or a fitness bracelet.

18. A method for determining a hemoglobin concentration in a user's blood, the method comprising:
irradiating a tissue of the user with radiation of at least two different wavelengths;
detecting photoplethysmographic (PPG) signals from the user's tissue in a time domain at the at least two different wavelengths in a reflection mode;
transforming the detected PPG signals from the time domain to a frequency domain or a time-frequency domain;
describing the transformed PPG signals with respect to harmonics to obtain a set of characteristics; and
determining a hemoglobin concentration in the user's blood based on the obtained set of characteristics and an information from a database.

19. The method of claim 18,
wherein, in the describing of the PPG signals:
the most significant signal is selected from all the PPG signals and is used as a reference signal,
the harmonics are extracted from the reference signal and are used as coordinates, and
all the PPG signals are decomposed according to the coordinates, and
wherein, in the determining of the hemoglobin concentration, the obtained set of characteristics is compared with sets of characteristics from the database, each of which corresponds to a specific value of hemoglobin concentration.

20. A system for determining a hemoglobin concentration in a user's blood, the system comprising:
the wearable device of claim 1; and
a remote server and/or a cloud storage,
wherein the system comprises means for a communication interface between the wearable device and the remote server and/or the cloud storage.
